SEQUENCE LISTING (137 sequences)

SEQ ID NO: 1 - protein, 15 aa, Artificial Sequence
  Synthetic
MNYALKGQGRTLYGF

SEQ ID NO: 2 - protein, 15 aa, Artificial Sequence
  Synthetic
SNYALKGQGRTLYGF

SEQ ID NO: 3 - protein, 15 aa, Artificial Sequence
  Synthetic
TNYALKGQGRTLYGF

SEQ ID NO: 4 - protein, 15 aa, Artificial Sequence
  Synthetic
GNYALKGQGRTLYGF

SEQ ID NO: 5 - protein, 15 aa, Artificial Sequence
  Synthetic
LNYALKGQGRTLYGF

SEQ ID NO: 6 - protein, 15 aa, Artificial Sequence
  Synthetic
ANYALKGQGRTLYGF

SEQ ID NO: 7 - protein, 15 aa, Artificial Sequence
  Synthetic
VNYALKGQGRTLYGF

SEQ ID NO: 8 - protein, 15 aa, Artificial Sequence
  Synthetic
INYALKGQGRTLYGF

SEQ ID NO: 9 - protein, 15 aa, Artificial Sequence
  Synthetic
FNYALKGQGRTLYGF

SEQ ID NO: 10 - protein, 15 aa, Artificial Sequence
  Synthetic
YNYALKGQGRTLYGF

SEQ ID NO: 11 - protein, 15 aa, Artificial Sequence
  Synthetic
CNYALKGQGRTLYGF

SEQ ID NO: 12 - protein, 12 aa, Artificial Sequence
  Synthetic
SNYALKGQGRTL

SEQ ID NO: 13 - protein, 12 aa, Artificial Sequence
  Synthetic
TNYALKGQGRTL

SEQ ID NO: 14 - protein, 12 aa, Artificial Sequence
  Synthetic
GNYALKGQGRTL

SEQ ID NO: 15 - protein, 12 aa, Artificial Sequence
  Synthetic
LNYALKGQGRTL

SEQ ID NO: 16 - protein, 12 aa, Artificial Sequence
  Synthetic
ANYALKGQGRTL

SEQ ID NO: 17 - protein, 12 aa, Artificial Sequence
  Synthetic
VNYALKGQGRTL

SEQ ID NO: 18 - protein, 12 aa, Artificial Sequence
  Synthetic
INYALKGQGRTL

SEQ ID NO: 19 - protein, 12 aa, Artificial Sequence
  Synthetic
FNYALKGQGRTL

SEQ ID NO: 20 - protein, 12 aa, Artificial Sequence
  Synthetic
YNYALKGQGRTL

SEQ ID NO: 21 - protein, 12 aa, Artificial Sequence
  Synthetic
MNYALKGQGRTL

SEQ ID NO: 22 - protein, 13 aa, Artificial Sequence
  Synthetic
CMNYALKGQGRTL

SEQ ID NO: 23 - protein, 10 aa, Artificial Sequence
  Synthetic
SNYALKGQGR

SEQ ID NO: 24 - protein, 10 aa, Artificial Sequence
  Synthetic
TNYALKGQGR

SEQ ID NO: 25 - protein, 10 aa, Artificial Sequence
  Synthetic
GNYALKGQGR

SEQ ID NO: 26 - protein, 10 aa, Artificial Sequence
  Synthetic
LNYALKGQGR

SEQ ID NO: 27 - protein, 10 aa, Artificial Sequence
  Synthetic
ANYALKGQGR

SEQ ID NO: 28 - protein, 10 aa, Artificial Sequence
  Synthetic
VNYALKGQGR

SEQ ID NO: 29 - protein, 10 aa, Artificial Sequence
  Synthetic
INYALKGQGR

SEQ ID NO: 30 - protein, 10 aa, Artificial Sequence
  Synthetic
FNYALKGQGR

SEQ ID NO: 31 - protein, 10 aa, Artificial Sequence
  Synthetic
YNYALKGQGR

SEQ ID NO: 32 - protein, 10 aa, Artificial Sequence
  Synthetic
MNYALKGQGR

SEQ ID NO: 33 - protein, 10 aa, Artificial Sequence
  Synthetic
CNYALKGQGR

SEQ ID NO: 34 - protein, 15 aa, Artificial Sequence
  Synthetic
RNYALKGQGRTLYGF

SEQ ID NO: 35 - protein, 12 aa, Artificial Sequence
  Synthetic
RNYALKGQGRTL

SEQ ID NO: 36 - protein, 10 aa, Artificial Sequence
  Synthetic
RNYALKGQGR

SEQ ID NO: 37 - protein, 15 aa, Artificial Sequence
  Synthetic
SRYALKGQGRTLYGF

SEQ ID NO: 38 - protein, 12 aa, Artificial Sequence
  Synthetic
SRYALKGQGRTL

SEQ ID NO: 39 - protein, 10 aa, Artificial Sequence
  Synthetic
SRYALKGQGR

SEQ ID NO: 40 - protein, 15 aa, Artificial Sequence
  Synthetic
SNYALKRQGRTLYGF

SEQ ID NO: 41 - protein, 12 aa, Artificial Sequence
  Synthetic
SNYALKRQGRTL

SEQ ID NO: 42 - protein, 10 aa, Artificial Sequence
  Synthetic
SNYALKRQGR

SEQ ID NO: 43 - protein, 14 aa, Artificial Sequence
  Synthetic
RYALKGQGRTLYGF

SEQ ID NO: 44 - protein, 11 aa, Artificial Sequence
  Synthetic
RYALKGQGRTL

SEQ ID NO: 45 - protein, 9 aa, Artificial Sequence
  Synthetic
RYALKGQGR

SEQ ID NO: 46 - protein, 14 aa, Artificial Sequence
  Synthetic
NYALKRQGRTLYGF

SEQ ID NO: 47 - protein, 11 aa, Artificial Sequence
  Synthetic
NYALKRQGRTL

SEQ ID NO: 48 - protein, 9 aa, Artificial Sequence
  Synthetic
NYALKRQGR

SEQ ID NO: 49 - protein, 13 aa, Artificial Sequence
  Synthetic
YALRGQGRTLYGF

SEQ ID NO: 50 - protein, 10 aa, Artificial Sequence
  Synthetic
YALRGQGRTL

SEQ ID NO: 51 - protein, 8 aa, Artificial Sequence
  Synthetic
YALRGQGR

SEQ ID NO: 52 - protein, 12 aa, Artificial Sequence
  Synthetic
ALRGQGRTLYGF

SEQ ID NO: 53 - protein, 9 aa, Artificial Sequence
  Synthetic
ALRGQGRTL

SEQ ID NO: 54 - protein, 7 aa, Artificial Sequence
  Synthetic
ALRGQGR

SEQ ID NO: 55 - protein, 11 aa, Artificial Sequence
  Synthetic
LRGQGRTLYGF

SEQ ID NO: 56 - protein, 8 aa, Artificial Sequence
  Synthetic
LRGQGRTL

SEQ ID NO: 57 - protein, 6 aa, Artificial Sequence
  Synthetic
LRGQGR

SEQ ID NO: 58 - protein, 10 aa, Artificial Sequence
  Synthetic
RGQGRTLYGF

SEQ ID NO: 59 - protein, 7 aa, Artificial Sequence
  Synthetic
RGQGRTL

SEQ ID NO: 60 - protein, 5 aa, Artificial Sequence
  Synthetic
RGQGR

SEQ ID NO: 61 - protein, 17 aa, Artificial Sequence
  Synthetic
SNYALKGQGRTLYGFGG

SEQ ID NO: 62 - protein, 17 aa, Artificial Sequence
  Synthetic
TNYALKGQGRTLYGFGG

SEQ ID NO: 63 - protein, 17 aa, Artificial Sequence
  Synthetic
GNYALKGQGRTLYGFGG

SEQ ID NO: 64 - protein, 17 aa, Artificial Sequence
  Synthetic
LNYALKGQGRTLYGFGG

SEQ ID NO: 65 - protein, 17 aa, Artificial Sequence
  Synthetic
ANYALKGQGRTLYGFGG

SEQ ID NO: 66 - protein, 17 aa, Artificial Sequence
  Synthetic
VNYALKGQGRTLYGFGG

SEQ ID NO: 67 - protein, 17 aa, Artificial Sequence
  Synthetic
INYALKGQGRTLYGFGG

SEQ ID NO: 68 - protein, 17 aa, Artificial Sequence
  Synthetic
FNYALKGQGRTLYGFGG

SEQ ID NO: 69 - protein, 17 aa, Artificial Sequence
  Synthetic
YNYALKGQGRTLYGFGG

SEQ ID NO: 70 - protein, 17 aa, Artificial Sequence
  Synthetic
MNYALKGQGRTLYGFGG

SEQ ID NO: 71 - protein, 17 aa, Artificial Sequence
  Synthetic
CNYALKGQGRTLYGFGG

SEQ ID NO: 72 - protein, 15 aa, Artificial Sequence
  Synthetic
VVYALKRQGRTLYGF

SEQ ID NO: 73 - protein, 15 aa, Artificial Sequence
  Synthetic
KNYALKGQGRTLYGF

SEQ ID NO: 74 - protein, 12 aa, Artificial Sequence
  Synthetic
KNYALKGQGRTL

SEQ ID NO: 75 - protein, 10 aa, Artificial Sequence
  Synthetic
KNYALKGQGR

SEQ ID NO: 76 - protein, 15 aa, Artificial Sequence
  Synthetic
SKYALKGQGRTLYGF

SEQ ID NO: 77 - protein, 12 aa, Artificial Sequence
  Synthetic
SKYALKGQGRTL

SEQ ID NO: 78 - protein, 10 aa, Artificial Sequence
  Synthetic
SKYALKGQGR

SEQ ID NO: 79 - protein, 15 aa, Artificial Sequence
  Synthetic
SNYALKKQGRTLYGF

SEQ ID NO: 80 - protein, 12 aa, Artificial Sequence
  Synthetic
SNYALKKQGRTL

SEQ ID NO: 81 - protein, 10 aa, Artificial Sequence
  Synthetic
SNYALKKQGR

SEQ ID NO: 82 - protein, 14 aa, Artificial Sequence
  Synthetic
KYALKGQGRTLYGF

SEQ ID NO: 83 - protein, 11 aa, Artificial Sequence
  Synthetic
KYALKGQGRTL

SEQ ID NO: 84 - protein, 9 aa, Artificial Sequence
  Synthetic
KYALKGQGR

SEQ ID NO: 85 - protein, 14 aa, Artificial Sequence
  Synthetic
NYALKKQGRTLYGF

SEQ ID NO: 86 - protein, 11 aa, Artificial Sequence
  Synthetic
NYALKKQGRTL

SEQ ID NO: 87 - protein, 9 aa, Artificial Sequence
  Synthetic
NYALKKQGR

SEQ ID NO: 88 - protein, 13 aa, Artificial Sequence
  Synthetic
YALKGQGRTLYGF

SEQ ID NO: 89 - protein, 10 aa, Artificial Sequence
  Synthetic
YALKGQGRTL

SEQ ID NO: 90 - protein, 8 aa, Artificial Sequence
  Synthetic
YALKGQGR

SEQ ID NO: 91 - protein, 12 aa, Artificial Sequence
  Synthetic
ALKGQGRTLYGF

SEQ ID NO: 92 - protein, 9 aa, Artificial Sequence
  Synthetic
ALKGQGRTL

SEQ ID NO: 93 - protein, 7 aa, Artificial Sequence
  Synthetic
ALKGQGR

SEQ ID NO: 94 - protein, 11 aa, Artificial Sequence
  Synthetic
LKGQGRTLYGF

SEQ ID NO: 95 - protein, 8 aa, Artificial Sequence
  Synthetic
LKGQGRTL

SEQ ID NO: 96 - protein, 6 aa, Artificial Sequence
  Synthetic
LKGQGR

SEQ ID NO: 97 - protein, 10 aa, Artificial Sequence
  Synthetic
KGQGRTLYGF

SEQ ID NO: 98 - protein, 7 aa, Artificial Sequence
  Synthetic
KGQGRTL

SEQ ID NO: 99 - protein, 5 aa, Artificial Sequence
  Synthetic
KGQGR

SEQ ID NO: 100 - protein, 16 aa, Artificial Sequence
  Synthetic
SNYALKGQGRTLYGFC

SEQ ID NO: 101 - protein, 16 aa, Artificial Sequence
  Synthetic
TNYALKGQGRTLYGFC

SEQ ID NO: 102 - protein, 16 aa, Artificial Sequence
  Synthetic
GNYALKGQGRTLYGFC

SEQ ID NO: 103 - protein, 16 aa, Artificial Sequence
  Synthetic
LNYALKGQGRTLYGFC

SEQ ID NO: 104 - protein, 16 aa, Artificial Sequence
  Synthetic
ANYALKGQGRTLYGFC

SEQ ID NO: 105 - protein, 16 aa, Artificial Sequence
  Synthetic
VNYALKGQGRTLYGFC

SEQ ID NO: 106 - protein, 16 aa, Artificial Sequence
  Synthetic
INYALKGQGRTLYGFC

SEQ ID NO: 107 - protein, 16 aa, Artificial Sequence
  Synthetic
FNYALKGQGRTLYGFC

SEQ ID NO: 108 - protein, 16 aa, Artificial Sequence
  Synthetic
YNYALKGQGRTLYGFC

SEQ ID NO: 109 - protein, 16 aa, Artificial Sequence
  Synthetic
MNYALKGQGRTLYGFC

SEQ ID NO: 110 - protein, 16 aa, Artificial Sequence
  Synthetic
CNYALKGQGRTLYGFC

SEQ ID NO: 111 - protein, 15 aa, Artificial Sequence
  Synthetic
VVYALKKQGRTLYGF

SEQ ID NO: 112 - protein, 14 aa, Artificial Sequence
  Synthetic
NYALKGQGRTLYGF

SEQ ID NO: 113 - protein, 9 aa, Artificial Sequence
  Synthetic
GQGRTLYGF

SEQ ID NO: 114 - protein, 8 aa, Artificial Sequence
  Synthetic
QGRTLYGF

SEQ ID NO: 115 - protein, 14 aa, Artificial Sequence
  Synthetic
SNYALKGQGRTLYG

SEQ ID NO: 116 - protein, 13 aa, Artificial Sequence
  Synthetic
SNYALKGQGRTLY

SEQ ID NO: 117 - protein, 15 aa, Artificial Sequence
  Synthetic
SHYALKGQGRTLYGF

SEQ ID NO: 118 - protein, 15 aa, Artificial Sequence
  Synthetic
QNYALKGQGRTLYGF

SEQ ID NO: 119 - protein, 15 aa, Artificial Sequence
  Synthetic
SAYALKGQGRTLYGF

SEQ ID NO: 120 - protein, 15 aa, Artificial Sequence
  Synthetic
SSYALKGQGRTLYGF

SEQ ID NO: 121 - protein, 15 aa, Artificial Sequence
  Synthetic
ENYALKGQGRTLYGF

SEQ ID NO: 122 - protein, 16 aa, Artificial Sequence
  Synthetic
SNYALKGQGRTLYGFG

SEQ ID NO: 123 - protein, 15 aa, Artificial Sequence
  Synthetic
SHYALKGQGRTLYGF

SEQ ID NO: 124 - protein, 9 aa, Artificial Sequence
  Synthetic
NYALKGQGR

SEQ ID NO: 125 - protein, 15 aa, Artificial Sequence
  Synthetic
MRYALKGQGRTLYGF

SEQ ID NO: 126 - protein, 15 aa, Artificial Sequence
  Synthetic
MKYALKGQGRTLYGF

SEQ ID NO: 127 - protein, 15 aa, Artificial Sequence
  Synthetic
XNYALKGQGRTLYGF

SEQ ID NO: 128 - protein, 15 aa, Artificial Sequence
  Synthetic
VVYALKRQGXTLYGF

SEQ ID NO: 129 - protein, 15 aa, Artificial Sequence
  Synthetic
VVYTLKRQGRTLYGF

SEQ ID NO: 130 - protein, 4 aa, Artificial Sequence
  Synthetic
RQGR

SEQ ID NO: 131 - protein, 4 aa, Artificial Sequence
  Synthetic
GQGR

SEQ ID NO: 132 - protein, 4 aa, Artificial Sequence
  Synthetic
GQAR

SEQ ID NO: 133 - protein, 4 aa, Artificial Sequence
  Synthetic
RQAR

SEQ ID NO: 134 - protein, 4 aa, Artificial Sequence
  Synthetic
GQYR

SEQ ID NO: 135 - protein, 5 aa, Artificial Sequence
  Synthetic
GQYXR

SEQ ID NO: 136 - protein, 6 aa, Artificial Sequence
  Synthetic
GXQYXR

SEQ ID NO: 137 - protein, 9 aa, Artificial Sequence
  Synthetic
RQGRTLYGF

(19) United States
(12) Patent Application Publication
(10) Pub. No.: US 2009/0124555 A1
(43) Pub. Date: May 14, 2009

(54) USE OF HISTOGRANIN AND HISTOGRANIN-LIKE COMPOUNDS AS INHIBITORS OF P2X7 RECEPTOR FUNCTION AND AS ANTI-ARTHRITIC AGENTS

(76) Inventors: Irma Bernatchez-Lemaire, Ottawa (CA); Simon Lemaire, Ottawa (CA)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(21) Appl. No.: 12/065,196

(22) PCT Filed: Aug. 23, 2006
(86) PCT No.: PCT/CA06/01382

§ 371 Date: Feb. 28, 2008

Related U.S. Application Data
(60) Provisional application No. 60/712,286, filed on Aug. 29, 2005.

(51) Int. Cl. A61K 38/08; A61K 38/00; A61P 19/00; A61P 11/00; A61P 25/28; A61P 29/00; A61P 9/10; C07K 7/02; C07K 7/08; C12Q 1/02

(52) U.S. Cl. 514/14; 514/15; 530/326; 435/29

(57) ABSTRACT

The invention relates to the use of Histogranin-like compounds to reduce P2X₇ (also designed P2Z) receptor function. The invention also provides a method for the prevention and treatment of rheumatoid arthritis and a variety of diseases/disorders including inflammatory disorders and neurodegenerative diseases.

### FIELD OF INVENTION

The present invention relates to Histogranin-like peptides and non-peptides for use as inhibitors of purinergic P2X₇ receptor-like activities and for the management of rheumatoid arthritis, inflammatory disorders and neurodegenerative diseases.

### BACKGROUND OF THE INVENTION

Nucleotides such as ATP accumulate at sites of inflammation and tissue injury and therefore may serve as paracrine regulators of cell function in inflammatory diseases and neurodegenerative disorders. The extracellular actions of ATP are mediated through a family of receptors termed P2 purinergic receptors. Cell surface ATP receptors or P2 receptors can be divided into two major classes: the metabotropic receptor family (P2Y/P2U) and the ionotropic receptor family (P2X). The metabotropic class belongs to the superfamily of G protein-coupled receptors. The ionotropic receptor family are ligand-gated channels. The latter comprises seven members (P2X₁ to P2X₇) which are ATP-gated ion channels. The P2X₇ receptor (originally known as P2Z) is a 595-amino acid polypeptide with two membrane-spanning domains and intracellular N- and C-terminal domains (North et al. (2000) Ann. Rev. Pharmacol. Toxicol. 40: 563-580; North (2003) Physiol. Rev. 82:1013-1067) which exhibits unique characteristics (features) that clearly distinguish it from the other members of the family. First higher concentrations of ATP are necessary to fully activate P2X₇ receptor (>1 mM compared to micromolar concentrations for other P2X receptors). Second, upon activation, the P2X₇ receptor has the unique ability to generate nonselective membrane pores that are permeable to low molecular weight solutes (<900 Da) (Steinberg et al. (1987) J. Biol. Chem. 262:8884-8886; Dubyak et al. (1993) Am. J. Physiol. 265: C577-C606; Surprenant et al. (1996) Science 272: 735-738). The carboxyterminal domain of P2X₇ receptor which is significantly longer than the comparable domains in the other P2X receptors participates in pore complex formation (North (1996) Curr. Opin. Cell. Biol. 8: 474-483). Third, BzATP is a more potent agonist for P2X₇ receptor activation than ATP and receptor activation is preferentially inhibited by oxidized ATP (oATP) (Murgia et al. (1993) J. Biol. Chem. 268:8199-8203; Di Virgilio (2003) Br. J. Pharmacol. 140:441-443) as well as KN-62 (in human cells) (Gargett et al. (1997) Br. J. Pharmacol. 120:1483-1490) and Brilliant Blue G (BBG) (in rat cells) (Jiang et al. (2000) Mol. Pharmacol. 58: 82-88). Fourth, activation of the P2X₇ receptor has been uniquely associated with a number of important biological activities including a) induction of apoptosis and cytotoxicity (possibly as a result of sustained pore opening (Murgia et al. (1992) Biochem. J. 288:897-901; Ferrari et al. (1999) FEBS Lett. 447:71-75), b) stimulation of multinucleated giant cell formation (a hallmark of chronic inflammation) (Falzoni et al. (1995) J. Clin. Invest. 95:1207-1216; Chiozzi et al. (1997) J. Cell Biol. 138:697-706) and c) induction of interleukin (IL)-1 posttranslational processing and release (Hogquist et al. (1991) Proc. Natl. Acad. Sci. (USA) 88:8485-8489; Perregaux et al. (1994) J. Biol. Chem. 269:15195-15203; Sanz et al. (2000) J. Immunol. 164:4893-4898; Solle et al. (2001) J. Biol. Chem. 276:125-132). Besides these activities that clearly distinguish P2X₇ receptor from the other members of the P2X family, the P2X₇ receptor also participates in various cellular activities including lymphocyte proliferation (Baricordi et al. (1999) J. Biol. Chem. 274: 33206-33208), fertilization (Foresta et al. (1996) Am. J. Physiol. C1709-C1714), killing of invading mycobacteria and some intracellular pathogens (Lammas et al. (1997) Immunity 7:433-444; Coutinho-Silva et al. (2003) Immunity 19:403-412), degranulation of mast cells and the shedding of surface molecules, L-selectin and CD 23 from lymphocytes (Gu et al. (1998) Blood 92: 946-951). Further, ligation of P2X₇ receptor has been associated with activation of phospholipase D (El-Moatassim et al. (1993) J. Biol. Chem. 268:15571-15578) and NF-kappaB, a transcription factor that controls cytokine expression and apoptosis (Ferrari et al. (1997) J. Cell Biol. 139:1635-1643).

One of the most interesting attribute of P2X₇ receptor is its ability to induce the posttranslational processing of precursor IL-1 (proIL-1) and the release of mature IL (Hogquist et al. (1991) Proc. Natl. Acad. Sci. (USA) 88:8465-8489; Perregaux et al. (1994) J. Biol. Chem. 269:15195-15203, Sanz et al. (2000) J. Immunol. 164:4893-4898) a multipotential inflammatory mediator produced mainly by monocytes and macrophages. Two distinct gene products. IL-1.beta and IL-1.alpha contribute to IL-1 biological activity. Despite significant difference in amino acid sequence (only 25% homology). IL-1.beta and IL-1.alpha bind to the same receptor on target cells (Slack et al. (1993) J. Biol. Chem. 268:2513-2524). Following cell activation, both IL-1.alpha and IL-1.beta are initially produced as 31 kDa precursor molecules that are subsequently cleaved into mature 17 kDa cytokines by proteolysis. In the case of IL-1.alpha, proteolytic cleavage is not necessary to generate a receptor-competent ligand and both proIL-1.alpha and the 17 kDa cleavage product display equivalent signaling activity. In contrast, proIL-1.beta does not bind to the signaling IL-1 receptor (Mosley et al. (1987) J. Biol. Chem. 262:2941-2944) and requires cleavage by caspase-1 to generate mature 17 kDa IL-1.beta, the bioactive form (Ceretti et al. (1992) Sciences 256:97-100; Thornberry et al. (1992) Nature 356:768-774). An important and unique attribute of both proIL-1. beta and proIL-1.alpha is their lack of a signal peptide (March et al. (1985) Nature 315:641-647) required to direct nascent polypeptides to the endoplasmic reticulum and the secretory apparatus (Walter et al. (1994) Ann. Rev. Cell Biol. 10:87-119), and this results in their accumulation within the cytoplasmic compartment. The mechanisms that control IL-1 processing and release are not well understood but recent studies have provided evidence that post-translational processing of proIL-1 requires that monocytes/macrophages activated by lipopolysaccharide (LPS) encounter a second stimulus that promotes efficient cleavage of prolL-1.beta and release of the 17-kDa bioactive cytokine (Miller et al. (1995) J. Immunol. 154:1331-1338; Laliberte et al. (1999) J. Biol. Chem. 274:36944-36951; Sanz et al. (2000) J. Immunol. 164:4893-4898). A number of stimuli including ATP, nigericin, cytolytic T-cells, bacterial toxins and hypotonic stress (Bhakdi et al. (1990) J. Clin. Invest. 85:7988-7992; Hogquist et al. (1991) Proc. Natl. Acad. Sci. (USA) 88:8485-8489; Perregaux et al. (1992) J. Immunol. 149:1294-1303; Perregaux et al. (1994) J. Biol. Chem. 269:15195-15203; Perregaux et al. (1996) J. Immunol. 157:57-64; Walev et al. (1995) EMBO J. 14:1607-1614) have been reported to promote in vitro IL-1 posttranslational processing by LPS-activated monocytes and/or macrophages. In particular. ATP challenge of LPS-activated macrophages stimulates the release of large quantities of IL-1.beta in cell cultures (Hogquist et al. (1991) Proc. Natl. Acad. Sci. (USA) 88:8485-8489: Perregaux et al. (1994) J. Biol. Chem. 269:15195-15203; Sanz et al. (2000) J. Immunol. 164:4893-4898). Further, extracellular release of IL-1.beta in vivo also requires ATP as a secretion stimulus; in response to intraperitoneal injection (i.p.) of LPS, mouse peritoneal macrophages produce proIL-1.beta but release little cytokine extracellularly. However, subsequent i.p. injection of ATP stimulates generation of large quantities of extracellular mature IL-1.beta (Griffiths et al. (1995) J. Immunol. 154:2821-2828). Most importantly, it was demonstrated unequivocally that such ATP effect is mediated selectively by the P2X₇ receptor since P2X₇^{-/-} knock-out mice primed i.p. with LPS and subsequently challenged with ATP, failed to release detectable levels of IL-1.beta and IL-1.alpha, and released reduced levels of IL-6 extracellularly (Solle et al. (2001) J. Biol. Chem. 276:125-132). In addition, P2X₇ receptor activation has been recently linked to IL-18 release (Perregaux et al. (2000) J. Immunol. 165:4615-4623; Mehta et al. (2001) J. Biol. Chem. 276:3820-3896; Sluyter et al. (2004) Genes Immun. 5:585-591). Therefore, P2X₇ receptor plays a crucial role in the release of potent pro-inflammatory cytokines such as IL-1.beta, IL-1.alpha, IL-6 and IL-18. Interestingly, the P2X₇ receptor has been shown to be present mainly in cells regulating inflammatory and immune responses in various tissues including macrophages, microglial cells (brain macrophages), lymphocytes, dendritic cells and mast cells (rev. in Di Virgilio et al. (2001) Blood 97:587-600; Bulanova et al. (2005) J. Immunol. 174:3880-3890). Such distribution of P2X₇ receptor coupled to its unique ability to induce the release of large quantities of potent pro-inflammatory cytokines point to a crucial role of P2X₇ receptor in the pathogenesis of many inflammatory diseases and the development of pain (Chessell et al. (2005) Pain 114:386-389), an unavoidable accompanying symptom of inflammation. When released from cells, IL-1 elicits complex signaling cascades leading to the upregulation of gene products that contribute to an inflammatory state including matrix metallo proteinases cyclooxygenase-9, IL-6 and cellular adhesion molecules (Flannery et al. (1999) J. Matrix Biol. 18:225-237; Guzu et al. (1998) J. Biol. Chem. 273:28670-28676; Allen et al. (2000) J. Exp. Med. 191:859-869; Bevilacqua et al. (1989) Science 243:1160-1164). Notably, the cytokine IL-1 plays a major role in a wide range of inflammatory and autoimmune diseases. These include but are not restricted to rheumatoid arthritis (RA), osteoarthritis (OA), chronic obstructive pulmonary disease (COPD), asthma, inflammatory bowel disease (IBD) including Crohn's disease (CD) and ulcerative colitis (UC), atherosclerosis and diseases of the central nervous system such as multiple sclerosis (MS). Alzheimer's disease and stroke. A pro-inflammatory, tissue-destructive role for IL-1 has been implicated in many human diseases (Arend et al. (1998) Annu. Rev. Immunol. 16:27-55; Hallegua et al. (2003) Ann. Rheum. Dis. 61:960-967; Cominelli et al. (1996) Aliment. Pharmacol. Ther. 10:49-53; Rothwell et al. (2003) Brain Behav. Immun. 17:152-157; Dayer (2002) Joint Bone Spine 69:123-132; Lappalainen et al. (2005) Am. J. Respir. Cell Mol. Biol. 32:311-318). Increased production of IL-1.beta, or the naturally occurring IL-1 receptor antagonist (L-1Ra) has been demonstrated in the circulation of patients with RA (Chikanza et al. (1995) Arthritis Rheum. 38:642-648) and IBD (Casini-Raggi et al. (1995) J. Immunol. 154:2434-2440) and the levels of measured IL-1.beta have been shown to correlate with disease severity in RA (Eastgate et al. (1988) Lancet 2:706-708; Rooney et al. (1990) Rheumatol. Int. 10:217-219), COPD (Dentener et al. (2001) Thorax 56:721-726) and UC (Cominelli et al. (1996) Aliment. Pharmacol. Ther. 10:49-53; Casini-Raggi et al. (1995) J. Immunol. 154:2434-2440; Isaacs et al. (1992) Gastroenterology 103:1587-1595). Elevated levels of IL-1.beta have been detected in the synovium, synovial fluid, cartilage and branchoalveolar lavage of patients with RA (Firestein et al. (1992) Arthritis Rheum. 149:1054-1062; Kahle et al. (1992) Ann. Rheum. Dis. 51:731-734; Koch et al. (1992) Clin. Immunol. Immunopathol. 65:23-29), OA (Firestein et al. (1992) Arthritis Rheum. 149:1054-1062; Nouri et al. (1984) Clin. Exp. Immunol. 55:295-302), COPD (Chung (2001) Eur. Resp. J. Suppi. 34:S50-S59) and asthma (Broide et al. (1992) J. Allergy Clin. Immunol. 89:958-967; Sousa et al. (1996) Am. J. Respir. Crit. Care Med. 154:1061-1066). IL-1.beta mRNA and/or protein can be detected in tissue biopsies by polymerase chain reaction or immunohistochemistry in patients with UC and Crohn's disease (Isaacs et al. (1992) Gastroenterology 103:1587-1595), RA, OA, COPD, atherosclerosis (Pomerantz et al. (2001) Proc. Natl. Acad. Sci. (USA) 98:287-2876; Pearce et al. (1992) J. Vasc. Surg. 16:784-789). Alzheimer's disease (Lombardt et al. (1999) J. Neuroimmunol. 97:163-171; Griffin et al. (2002) J. Leukoc. Biol. 72:233-230: Sheng et al. (1995) Neuropathol. Appl. Neurobiol. 21:290-301), multiple sclerosis (Basu et al. (2002) J. Neurosci. 22:6071-6082) and sepsis (Calkins et al. (2002) J. Endotoxin Res. 8:59-67).

Recently, evidence for a direct implication of P2X₇ receptor in inflammatory diseases, inflammatory and neuropathic pain and neurodegenerative diseases have been obtained. Thus, P2X₇ receptor knock-out mice (P2X₇^{-/-}) showed a reduced severity of arthritis in an anti-collagen antibody arthritis model (Labasi et al. (2002) J. Immunol. 168:6436-6445). Moreover, disruption of the P2X₇ purinoceptor gene abolished chronic inflammatory and neuropathic pain (Chessell et al. (2005) Pain 114:386-389) and P2X₇ receptor was specifically up-regulated around β-amyloid plaques in a mouse model of Alzheimer's disease (Parvathenani et al. (2003) J. Biol. Chem. 278:13309-13317). Furthermore, the murine and human P2X₇ receptor genes lie within lupus susceptibility loci and it has been suggested that a reduction of P2X₇ receptor function may result in decreased severity of systemic lupus erythematosus (SLE) (Elliott et al. (2005) Arthritis Res. Ther. 7:R468-R475).

A need exists therefore to develop compounds effective as inhibitors of P2X₇ receptor-like activities for use in the treatment of chronic inflammatory and neurodegenerative diseases where the P2X₇ receptor may mediate cell death, tissue injury and the release of IL-1.beta. Few classes of P2X₇ antagonists are known in the literature. KN-62, a bi-isoquinolinesulphonyltyrosine derivative (Gargett et al. (1997) Br. J. Pharmacol, 120:1483-1490), potent analogues such as MRS 2306 (Ravi et al. (2001) Drug Dev. Res. 54:75-87), and phenylpiperazine derivatives (Baraldi et al. (2003) J. Med. Chem. 46:1318-1329) have been reported. These compounds have a large molecular weight (>700), are very lipophilic and because the amide, sulphonamide and sulphonate are all essential for potency, the transformation of KN-62 analogues into compounds with drug-like properties (Lipinski et al. (1998) Adv. Drug Deliv. Rev. 23:3-25) would be very challenging. A new class of adamantane amide (Baxter et al. (2003) Biorg. Med. Chem. Lett. 13:4047-4050) and cyclic imides (Alcaraz et al. (2003) Bioorg. Med. Chem. Lett. 13:4043-4046) has been recently reported as potent P2X₇ receptor antagonists based on their ability to inhibit in vitro the P2X₇-pore forming activity. Examples of compounds which act as P2X₇ receptor antagonists can be found in U.S. Pat. Application No. 20030040513; U.S. Pat. Application No. 20030181452, each incorporated by reference.

Attempts to synthesize small, synthetic inhibitors or antagonists of IL-1 have so far failed but potential therapeutic targets include modifiers of IL-1 expression and release (Braddock et al. (2004) Nat. Rev. Drug Discovery 3:1-10). In that category, diarylsulphonyl urea CP424174 and CP41245 have been shown to inhibit post-translational processing and release of IL-1.beta (Perregaux et al. (2001) J. Pharmacol. Exp. Ther. 299:187-197). These compounds termed cytokine-release inhibitory drugs (CRIDs) are not inhibitors of ATP-stimulated P2X₇ purinoceptor (Walev et al. (1995) EMBO J. 14:1607-1614) and their precise mechanism of action remains unknown.

Although the potential uses of these compounds have been implicated in numerous diseases, the disease types are named as a result of speculation without examples or analysis (for example, U.S. Pat. Application No. 20030040513; U.S. Pat. Application No. 20030181452). The effect and efficacy of these compounds in vivo on disease progression for the many diseases have not been addressed. Therefore, the outcome and consequence of the use of these inhibitors on disease progression in vivo remain unknown.

The present work relates to HN-like molecules that have a different chemical composition than the currently known P2X₇ receptor antagonists and inhibitors of IL-1 production.

### SUMMARY OF THE INVENTION

It is an object of the invention to provide an improved use of histogranin and histogranin-like compounds as inhibitors of P2X₇ receptor function and as anti-arthritic agents.

The present invention relates to the use of Histogranin compounds to reduce P2X₇ receptor-like activities in an animal cell and in a mammal, and provides a method for the treatment of rheumatoid arthritis, inflammatory diseases and neurodegenerative disorders.

In one aspect, the invention provides the use of Histogranin compounds for reducing P2X₇ receptor-like activities in animal cells including but not restricted to, macrophages. In one embodiment, cells incubated with Histogranin compounds exhibit reduced P2X₇ receptor-stimulated pore formation and intracellular translocation of macromolecules.

In some embodiments. Histogranin compounds reduce P2X₇ receptor-induced apoptosis and cell death. Incubation of animal cells with Histogranin compounds decreases the number of apoptotic cells and the extent of DNA breaks in response to ATP and inhibit ATP-induced release of the cytoplasmic enzyme lactate dehydrogenase (LDH).

In other embodiments, Histogranin compounds reduced P2X₇-stimulated interleukin-(IL)-1. alpha, IL-1. beta, or IL-18 production, or IL-6 release. The LPS-dependent release of IL-1 is an inefficient process and leads to little accumulation of extracellular cytokine. ATP acting at the P2X₇ receptor is needed as a second stimulus to elicit cytokine release. In this method, macrophages primed with LPS and exposed subsequently to ATP in the presence of Histogranin compounds, exhibit reduced release of extracellular IL-1.beta, IL-1.alpha and IL-6 as compared to macrophages treated with LPS and ATP.

In other embodiments, Histogranin compounds inhibit in animal cells ATP-induced translocation of phosphatidylserine from the inner leaflet of the plasma membrane to the outer (PS FLIP), an event associated with microvesicle shedding and rapid release of IL-1.beta, and mediated by P2X₇ receptor stimulation.

The invention provides, in another aspect, a method for reducing P2X₇-stimulated IL-1.beta, IL-1.alpha, and IL-18 production and IL-6 release in mammals. ATP is required in vivo as a secretion stimulus for the generation of extracellular release of IL-1.beta. IL-1.alpha and partly for IL-6 release in animals treated with LPS, and its action is mediated selectively by the P2X₇ receptor. In this method, initial i.p. injection of mice with LPS followed by subsequent i.p. injections of Histogranin-like compounds and ATP result in a significant reduction in the generation of extracellular IL-1.beta. IL-1.alpha and IL-6 in peritoneal lavage fluids as compared to the animals treated with LPS and ATP only. In other application, HN compounds can be used to decrease cytokine production by inflammatory cells that cause symptoms to develop in inflammatory diseases and neurodegenerative disorders.

In another aspect, the invention provides a new method for the treatment of rheumatoid arthritis (RA). In this method, Histogranin compounds are injected i.p. during the development of collagen-induced arthritis (CIA) in mice. In some embodiments of the method of the invention. i.p. treatment with Histogranin compounds before the onset of RA, reduces the clinical score of RA i.e. joint tissue swelling and oedema (redness), and paw thickness.

In some embodiments, i.p. treatment with Histogranin compounds reduces the histological changes associated with inflammatory arthritis notably soft tissue infiltrate, synovitis and joint exudates and in other embodiments, i.p. injection of Histogranin compounds reduces the histological changes associated with destructive arthritis including pannus formation, cartilage degradation and bone erosion.

In other embodiments, i.p. treatment with Histogranin compounds after the onset of RA, decreases the clinical score index of RA i.e. tissue swelling and oedema (redness) and paw thickness.

In other embodiments, i.p. injection of Histogranin compounds, reduces IL-1.beta levels in joints of CIA mice. HN compounds also reduce in these mice P2X₇-stimulated IL-1.beta in peripheral blood.

This summary of the invention does not necessarily describe all features of the invention.

### BRIEF DESCRIPTION OF THE DRAWINGS

These and other features of the invention will become more apparent from the following description in which reference is made to the appended drawings wherein:

FIGS. 1A-B shows the inhibition of P2X₇ receptor-induced membrane permeabilization and pore formation in macrophages by Histogranin compounds. (A) Alveolar macrophages isolated from normal rats were incubated with ATP, BzATP or UTP and the fluorescent dye ethidium bromide (EtBr) in the presence or absence of oxidized ATP (oATP). Brilliant blue G (BBG) or Histogranin compounds as indicated; results are expressed as the percent maximum uptake of EtBr in response to ATP or Bz ATP. n=4-55 for each group. (B) Concentration-response curves for Histogranin compounds C-14 (circle), C-4 (square) and C-15 (diamond) inhibition of ethidium bromide uptake in response to ATP: results are plotted as the percent of maximum uptake of EtBr in response to ATP. N=3-5 for each group.

FIG. 2A-C shows the inhibition of P2X₇ receptor-induced macrophage apoptosis and cell death by Histogranin compounds. (A) Morphologic assessment of apoptosis following dual staining with Hoechst and propidium iodide. Alveolar macrophages were incubated for 3 h with or without ATP in the presence and absence of HN compound C-4; apoptosis (increased nuclei fluorescence) without secondary necrosis (i.e. without nuclei labeled with propidium iodide) was evaluated by microscopy and results are expressed as percent apoptotic macrophages. n=4. (B) Detection of internucleosomal degradation of genomic DNA occurring during apoptosis. Alveolar macrophages were incubated with ATP (5 mM) in the presence and absence of various Histogranin compounds as indicated for 30 minutes. The medium was then replaced with fresh medium, and after 24 h incubation, cytoplasmic oligonucleosomal DNA was detected by ELISA; results are expressed as the percent maximum cytoplasmic oligonucleosomal DNA in response to ATP. n=3-5 for each group. (C) Cell death-associated LDH release. Alveolar macrophages were stimulated with LPS for 2 h followed by a 1 h incubation with ATP (5 mM) or a 30 minutes incubation with Nigericin (20 μM), a P2X₇-independent stimulus of cell death, in the presence and absence of HN compound C-4 (10⁻⁹ M). LDH was measured in cell-free culture supernatants using a cytotoxicity kit for LDH. Results are expressed as the percent of LDH release from cells. N=3.

FIG. 3A-C shows the inhibition of P2X₇ receptor-induced IL-1.beta release by HN compounds in macrophages. (A) Alveolar macrophages were incubated with LPS (1 μg/ml) or PBS for 3 h. Cells were then stimulated for 1 h with ATP (5 mM) or BzATP (0.5 mM) in the presence and absence of either Brilliant Blue G (BBG. 5×10⁻⁹M), a P2X₇ receptor antagonist. HN compound C-4 and HN compound C-15. Macrophages were also pre-treated for 2 h with oxidized ATP (oATP, 100 μM) an irreversible antagonist of P2X₇ receptor before the combined LPS and ATP stimulation. Cell-free culture supernatants were collected by centrifugation and IL-1.beta levels were measured by ELISA. Results are expressed as ng/ml. N=3-13 for each group. (B) Concentration-response curves for Histogranin compounds C-4 (square) and C-15 (circle) inhibition of ATP-induced release of Il-1.beta in macrophages. Cells were stimulated first with LPS followed by ATP as described in A, in the presence and absence of various concentrations of HN compounds, and levels of IL-1.beta in cell-free supernatants were measured by ELISA. Results are expressed as the percent maximum release of IL-1.beta in response to ATP. N=3. (C) Histogranin compound C-4 inhibits ATP- but not Nigericin-induced IL-1.beta release in macrophages. Cells were incubated for 3 h with LPS (1 Ξg/ml) followed by either ATP (5 mM) for 1 h or Nigericin (20 μM) for 30 minutes in the presence and absence of HN compound C-4. Levels of IL-1.beta were measured in cell-free culture supernatants by ELISA and results are expressed as the percent maximum release in response to ATP or Nigericin. N=3-4 for each group.

FIG. 4A-B shows the inhibition of P2X₇ receptor-induced IL-1.alpha release by HN compound C-4 in macrophages. (A) Alveolar macrophages were incubated with LPS or PBS for 3 h. Cells were then stimulated with ATP (5 mM) or BzATP (0.5 mM) for 1 h in the presence and absence of Histogranin compound C-4 (10⁻⁹M). Levels of IL-1.alpha were measured in cell-free culture supernatants by ELISA and results are expressed as ng/ml. N=4-5 for each group. (B) Concentration-response curves for Histogranin C-4 inhibition of ATP-(circle) and BzATP-(square) induced release of IL-1.alpha in macrophages. Cells were incubated with LPS for 3 h and ATP or BzATP was added as a secretion stimulus for 1 h in the presence and absence of various concentrations of HN compound C-4. Levels of IL-1.alpha were measured in cell-free culture supernatants by ELISA and results are expressed as percent maximal release of IL-1.alpha in response to ATP or Bz ATP. N=3

FIG. 5 shows the inhibition of P2X₇ receptor-induced Phosphatidylserine (PS) translocation (PS Flip) by Histogranin compound C-4 in macrophages. Alveolar macrophages were incubated in the presence and absence of HN compound C-4 (10⁻⁹M, and 10⁻⁸M) for 5 minutes. Cells were then labeled with fluorescein-conjugated annexin-V prior to stimulation with BzATP (100 μM), a potent P2X₇ receptor agonist for 5 minutes. Annexin-positive cells were analyzed by microscopy and capture image analysis, and results are expressed as percent of annexin-V positive cells. Control represents cells labeled with fluorescein-conjugated annexin-V for 5 minutes and incubated in the absence of BzATP for 5 minutes. N=4

FIG. 6A-B shows the inhibition of P2X₇ receptor-induced IL-1.beta release in mice treated with HN compounds. (A) Mice were injected intraperitoneally with LPS (50 μg). Two hours later, mice were injected i.p. with HN compound C-4 (0.1 mg or 0.3 mg) or PBS 15 minutes before the i.p. ATP challenge (5 mM). After 30 minutes, the peritoneal cavity was lavaged and the amount of IL-1.beta recovered in cell-free peritoneal lavage fluids was measured by ELISA. Results are expressed as pg/ml. N=10-13 for each group. (B) Inhibition of P2X₇ receptor-induced IL-1.beta release in mice treated with various HN compounds. Mice were injected i.p. with LPS and 2 h later with either PBS or ATP as described above in A. In parallel groups, mice were treated i.p. with one of the following RN compounds (C-1, C-4, C-5, C-6, C-9, C-10, C-11, C-12, C-13, C-14 or C-15) at doses of 0.1 mg and 0.3 mg, 15 minutes before the ATP injection. Cell-free supernatants of peritoneal lavage samples were tested for IL-1.beta by ELISA. Results are expressed as the percent maximum release of IL-1.beta in response to ATP stimulation. N=4-11 for each set group.

FIG. 7 shows the inhibition of P2X₇ receptor-induced IL-1.alpha release in mice treated with RN compound C-4. Mice were injected i.p. with LPS (50 μg) and 2 h later with either PBS or ATP (5 mM). In parallel, a group of mice were treated i.p. with 0.1 mg or 0.3 mg of HN compound C-4 15 minutes prior to the ATP injection. Thirty minutes later, mice were sacrificed and the amount of IL-1.alpha recovered in cell-free peritoneal lavage fluid was measured by ELISA. Results are expressed as pg/ml. N=5-7 for each group.

FIG. 8 shows the inhibition of P2X₇ receptor-induced IL-6 release in mice treated with HN compound C-4. Mice were injected i.p. with LPS (50 μg) and 2 h later with either PBS or ATP (5 mM). In parallel, a group of mice was treated i.p. with 0.1 mg or 0.3 mg of HN compound C-4 15 minutes prior to the ATP injection. Two hours later, mice were sacrificed and the amount of IL-6 recovered in cell-free peritoneal lavage fluid was measured by ELISA. Results are expressed as ng/ml. N=4-5 for each group.

FIG. 9A-C shows the reduction of limb swelling and clinical score index during collagen-induced arthritis in mice following treatment with HN compound C-4. (A) Groups of mice were injected with saline (negative control), collagen and LPS (positive control), collagen followed by i.p. daily treatment with RN compound C-4 (0.1 mg and 0.3 mg) before subsequent i.p. injection of LPS and onset of arthritis. Paw thickness was measured daily from day 44 to day 51 postimmunization with collagen. N=3-6 for each group. (B) Each limb of mice in various groups described in A, were monitored daily by macroscopic clinical scoring on a 0-3 scale. Results are expressed as clinical score index with a possible maximal clinical score of 12 per animal. N=3-10 for each group. (C) Groups of mice were injected with saline (negative control), collagen and LPS (positive control), collagen and LPS followed by i.p. daily injection of HN compound C-4 (0.3 mg) after onset of arthritis and the presence of a positive score index. Clinical scoring on a 0-3 scale was monitored daily up to 15 days post treatment with HN compound C-4. N=3-4 for each group.

FIG. 10 shows the reduction of histologic score of inflammatory and destructive arthritis during collagen-induced arthritis in mice following treatment with HN compound C-4. Groups of mice were injected with either saline (negative control), collagen and LPS (positive control), or collagen followed by i.p. daily treatment with HN compound C-4 (0.1 mg and 0.3 mg) before i.p. injection of LPS and onset of arthritis. Histologic assessment of arthritis was performed on knee sections graded 0 (normal) to 3 (severe) for six components of arthritis: soft tissue infiltrate/inflammation, synovitis, joint exudates, pannus, cartilage damage and bone destruction. N=3-5 for each group.

FIGS. 11A-B shows the treatment with HN compound C-4 reduces both clinical score index and IL-1.beta levels in paws of mice with collagen-induced arthritis. (A) Groups of mice were injected with either saline (negative control), collagen and LPS (positive control), or collagen followed by i.p. daily treatment with RN compound C-4 (0.1 mg and 0.3 mg) before i.p. injection of LPS and onset of arthritis. The front right paw of mice from various groups was evaluated by macroscopic clinical scoring on a 0-3 scale at sacrifice. Results are expressed as clinical score index with a possible maximal clinical score of 3 per paw. N=3-10 for each group. (B) After sacrifice, the front right paw of mice from various groups evaluated as described in A were snap frozen in liquid nitrogen and tissue lysates were analyzed for their IL-1.beta content by ELISA. Results are expressed as pg/mg protein.

FIG. 12A-C shows the treatment with various HN compounds reduces both clinical score index and P2X₇ receptor-stimulated IL-1 release in peripheral blood ex vivo. (A) Groups of mice were injected with either saline (negative control), collagen and LPS (positive control), or collagen followed by i.p. daily treatment with HN compounds C-1, C-9 or C-11 (0.3 mg) before i.p. injection of LPS and onset of arthritis. Each limb of mice in various groups was monitored daily by macroscopic clinical scoring on a 0-3 scale. Results are expressed as clinical score index with a possible maximal clinical score of 12 per animal. N=5-7 for each group. (B) Histologic assessment of arthritis was performed on both knees from mice in various groups as described in A. Knee sections were graded 0 (normal) to 3 (severe) for six components of arthritis: soft tissue infiltrate/inflammation, synovitis, joint exudates, pannus, cartilage damage and bone destruction. Results are expressed as histological score with a possible maximal score of 6 per animal. N=3-7 for each group. (C) P2X₇ receptor-stimulated IL-1.beta release in the periphery ex vivo was determined with a blood-based IL-1.beta assay. Blood was collected from mice injected either with saline (negative control), collagen and LPS (positive control) or collagen followed by i.p. daily treatment with HN compounds C-4 and C-9 before i.p. injection of LPS and onset of arthritis. Blood samples were incubated with and without LPS (1 μg/ml) for 3 h. ATP (5 mM, pH 7) was then added as a secretion stimulus and the mixtures were incubated for an additional 2 h. After centrifugation the resulting plasma samples were assayed for their IL-1.beta content by ELISA. Results are expressed as ng/ml. N=5-7 for each group.

### DETAILED DESCRIPTION

For the first time. Histogranin compounds are shown to reduce P2X₇ receptor-like activities in animal cells and in mammals, and to reduce symptoms and histopathological changes of RA.

By “Histogranin compounds” is meant Histogranin (Eur. J. Pharmacol. 1993 May 15; 945(3): 247-56). Histogranin-linear peptides (Can. J. Physiol. Pharmacol. 1995 February; 73(2); CA2097533. Histogranin-like cyclic tetrapeptides PCT/CA1998/01002; CA2,3006754; EP 98951127.4; U.S. Pat. No. 6,566,327; U.S. Pat. No. 6,855,6929 U.S. application Ser. No. 10/068,905: PCT/CA2003/00148; CA 2,475,609; EP 03737222.4; Prov. U.S. Application No. 60/472,780) and Histogranin-like non peptides (U.S. application Ser. No. 10/068,905; PCT/CA2003/00148; CA 2,475,609; EP 03737222.4). It also encompasses the Histogranin linear peptide analogs C-5 ([Val¹, Val², Arg⁷, [S-(3-nitro-2-sulfenyl pyridine)]Cys¹⁰]HN) and C-6 [Val¹, Val², Thr⁴, Arg⁷]HN, and the Histogranin-like cyclic peptide C-9 (Cyclic [Ala⁸, Tyr⁹, D-Arg¹⁰]HN-7-10 SL-108. Methods of preparing these compounds are included in the references cited above. All above references are incorporated herein by reference. Synthesis of C-5 and C-6 was performed as previously described (Prasad et al., Can J. Physiol. Pharmacol. 73:209-214, 1995, herein incorporated by reference) by the use of preformed symmetrical anhydrides of Boc-amino acids with the solid-phase method (Merrifield, J. Am. Chem. Soc. 85:2149-2154, 1963, herein incorporated by reference) and chloromethylated copolystyrene-divinylbenzene 1% crosslinkage (0.6 mequiv. Cl/g) as the resin. Peptide C-5: The C-terminal amino acid (Boc-Phe) was esterified to the resin according to the method of Gisin (Helv. Chim. Acta 56:1476-1482, 1973, herein incorporated by reference) with a final yield of 0.3 mmol/g resin. Thereafter, Boc-Gly, Boc-(2,6-diCl-Bzl)-Tyr, Boc-Leu, Boc-(Bzl)Thr, Boc-(NPys)Cys, Boc-Gly, Boc-Gln, Boc-(Tos)Arg, Boc-(2-Cl-Z)Lys, Boc-Leu,Boc-Ala, Boc-(2,6-diCl-Bzl)-Tyr, Boc-Val and Boc-Val were attached consecutively according to the following procedure: 1) one wash with CH₂Cl₂; 2) one pre-wash with 40% TFA in CH₂Cl₂; 3) 15 min deprotection with 40% TFA in CH₂Cl₂; 4) 3 washes with CH₂Cl₂; 5) one pre-wash with 5% DEA in CH₂Cl₂; 6) five min neutralization with 5% DEA in CH₂Cl₂; 7) three washes with CH₂Cl₂; 8) one hour coupling with preformed symmetrical anhydride of corresponding Boc-amino acid (six equivalents as compared with the resin substitution of 0.8 mmol/g); 9) three washes with CH₂Cl₂; 10) two washes with isopropanol. The peptide was cleaved from the resin and deprotected with liquid HF (15 ml) in the presence of anisole (10% v/v). HF was removed in vacuo and the residue was washed with ether before extraction of the peptide with 50% acetic acid followed by evaporation and filtration through a column of Sephadex G-10. The peptide fraction (ninhydrin detection on thin layer plate) was collected, lyophilized and submitted to purification by reversed phase HPLC on μ-Bondapak C-18 (Waters). The major peptide fraction was detected by UV at 240 nm and lyophilized to yield 90-95% of the pure compound (based on the starting Boc-Phe-resin). The purity and identity of the peptide were assessed by thin layer chromatography (TLC) on silica gel plates (BDH Chemical Associates of Merk Darmstadt, Germany) with the solvent system nBuOH:EtOH:HOAc:H₂O, 1:1:1:1; one spot) and mass spectrometry (M.W. 1870.89). C-6 was obtained by the same technique with the exception that the introduction of Boc-(NPys)Cys and Boc-Ala in the sequence cycles was replaced by Boc-(Tos)Arg and Boc-(Bzl)Thr, respectively. The purity of C-6 was also verified by TLC (one spot) and mass spectrometry (M.W.: 1800.0).

The Histogranin linear peptides useful in the present invention have been defined in Canadian Patent application 2,097,533, which is incorporated herein by reference. Examples of said peptides have a structure according to the following general formula: H—R₁-Gln-Gly-Arg-R₂—CO—R₃ wherein: R₁ represents one structure selected from the group consisting of: X-Asn-Tyr-Ala-Leu-Lys-Gly, X being an hydroxyl-containing amino acid; Y-Asn-Tyr-Ala-Leu-Lys-Gly, Y being a hydrocarbon side chain-containing amino acid; Z-Asn-Tyr-Ala-Leu-Lys-Gly, Z being an aromatic amino acid, W-Asn-Tyr-Ala-Leu-Lys-Gly W being a sulfur-containing amino acid; U-Asn-Tyr-Ala-Leu-Lys-Gly; Ser-U-Tyr-Ala-Leu-Lys-Gly; Ser-Asn-Tyr-Ala-Leu-Lys-U; U-Tyr-Ala-Leu-Lys-Gly; Asn-Tyr-Ala-Leu-Lys-U; Tyr-Ala-Leu-U-Gly; Ala-Leu-U-Gly; Leu-U-Gly; U-Gly; and Val-Val-Tyr-Ala-Leu-Lys-U-, U being a basic amino acid; R₂ represents one structure selected from the group consisting of: a single covalent bond (no intervening amino acids); Thr-Leu; Thr-Leu-Tyr-Gly-Phe; Thr-Leu-Tyr-Gly-Phe-Cys and Thr-Leu-Tyr-Gly-Phe-Gly-Gly; and R₃ represents a radical selected from the group consisting of —OH and —NH₂.

Examples of linear peptides useful to reduce P2X₇ receptor-like activities in an animal cell and in a mammal include the following:

| | |
|---|---|
| HN | [SEQ ID NO:1] |
| HN-amide | [SEQ ID NO:1] |
| [Ser¹]HN | [SEQ ID NO:2] |
| [Ser¹]HN-amide | [SEQ ID NO:2] |
| [Thr¹]HN | [SEQ ID NO:3] |
| [Thr¹]HN-amide | [SEQ ID NO:3] |
| [Gly¹]HN | [SEQ ID NO:4] |
| [Gly¹]HN-amide | [SEQ ID NO:4] |
| [Leu¹]HN | [SEQ ID NO:5] |
| [Leu¹]HN-amide | [SEQ ID NO:5] |
| [Ala¹]HN | [SEQ ID NO:6] |
| [Ala¹]HN-amide | [SEQ ID NO:6] |
| [Val¹]HN | [SEQ ID NO:7] |
| [Val¹]HN-amide | [SEQ ID NO:7] |
| [Ile¹]HN | [SEQ ID NO:8] |
| [Ile¹]HN-amide | [SEQ ID NO:8] |
| [Phe¹]HN | [SEQ ID NO:9] |
| [Phe¹]HN-amide | [SEQ ID NO:9] |
| [Tyr¹]HN | [SEQ ID NO:10] |
| [Tyr¹]HN-amide | [SEQ ID NO:10] |
| [Cys¹]HN | [SEQ ID NO:11] |
| [Cys¹]HN-amide | [SEQ ID NO:11] |
| [Ser¹]HN-(1-12) | [SEQ ID NO:12] |
| [Ser¹]HN-(1-12)-amide | [SEQ ID NO:12] |
| [Thr¹]HN-(1-12) | [SEQ ID NO:13] |
| [Thr¹]HN-(1-12)-amide | [SEQ ID NO:13] |
| [Gly¹]HN-(1-12) | [SEQ ID NO:14] |
| [Gly¹]HN-(1-12)-amide | [SEQ ID NO:14] |
| [Leu¹]HN-(1-12) | [SEQ ID NO:15] |
| [Leu¹]HN-(1-12)-amide | [SEQ ID NO:15] |
| [Ala¹]HN-(1-12) | [SEQ ID NO:16] |
| [Ala¹]HN-(1-12)-amide | [SEQ ID NO:16] |
| [Val¹]HN-(1-12) | [SEQ ID NO:17] |
| [Val¹]HN-(1-12)-amide | [SEQ ID NO:17] |
| [Ile¹]HN-(1-12) | [SEQ ID NO:18] |
| [Ile¹]HN-(1-12)-amide | [SEQ ID NO:18] |
| [Phe¹]HN-(1-12) | [SEQ ID NO:19] |
| [Phe¹]HN-(1-12)-amide | [SEQ ID NO:19] |
| [Tyr¹]HN-(1-12) | [SEQ ID NO:20] |
| [Tyr¹]HN-(1-12)-amide | [SEQ ID NO:20] |
| HN-(1-12) | [SEQ ID NO:21] |
| HN-(1-12)-amide | [SEQ ID NO:21] |
| [Cys¹]HN-(1-12) | [SEQ ID NO:22] |
| [Cys¹]HN-(1-12)-amide | [SEQ ID NO:22] |
| [Ser¹]HN-(1-10) | [SEQ ID NO:23] |
| [Ser¹]HN-(1-10)-amide | [SEQ ID NO:23] |
| [Thr¹]HN-(1-10) | [SEQ ID NO:24] |
| [Thr¹]HN-(1-10)-amide | [SEQ ID NO:24] |
| [Gly¹]HN-(1-10) | [SEQ ID NO:25] |
| [G1y¹]HN-(1-10)-amide | [SEQ ID NO:25] |
| [Leu¹]HN-(1-10) | [SEQ ID NO:26] |
| [Leu¹]HN-(1-10)-amide | [SEQ ID NO:26] |
| [Ala¹]HN-(1-10) | [SEQ ID NO:27] |
| [Ala¹]HN-(1-10)-amide | [SEQ ID NO:27] |
| [Val¹]HN-(1-10) | [SEQ ID NO:28] |
| [Val¹]HN-(1-10)-amide | [SEQ ID NO:28] |
| [Ile¹]HN-(1-10) | [SEQ ID NO:29] |
| [Ile¹]HN-(1-10)-amide | [SEQ ID NO:29] |
| [Phe¹]HN-(1-10) | [SEQ ID NO:30] |
| [Phe¹]HN-(1-10)-amide | [SEQ ID NO:30] |
| [Tyr¹]HN-(1-10) | [SEQ ID NO:31] |
| [Tyr¹]HN-(1-10)-amide | [SEQ ID NO:31] |
| HN-(1-10) | [SEQ ID NO:32] |
| HN-(1-10)-amide | [SEQ ID NO:32] |
| [Cys¹]HN-(1-10) | [SEQ ID NO:33] |
| [Cys¹]HN-(1-10)-amide | [SEQ ID NO:33] |
| [Arg¹]HN | [SEQ ID NO:34] |
| [Arg¹]HN-amide | [SEQ ID NO:34] |
| [Arg¹]HN-(1-12) | [SEQ ID NO:35] |
| [Arg¹]HN-(1-12)-amide | [SEQ ID NO:35] |
| [Arg¹]HN-(1-10) | [SEQ ID NO:36] |
| [Arg¹]HN-(1-10)-amide | [SEQ ID NO:36] |
| [Ser¹, Arg²]HN | [SEQ ID NO:37] |
| [Ser¹, Arg²]HN-amide | [SEQ ID NO:37] |
| [Ser¹, Arg²]HN-(1-12) | [SEQ ID NO:38] |
| [Ser¹, Arg²]HN-(1-12)-amide | [SEQ ID NO:38] |
| [Ser¹, Arg²]HN-(1-10) | [SEQ ID NO:39] |
| [Ser¹, Arg²]HN-(1-10)-amide | [SEQ ID NO:39] |
| [Ser¹, Arg⁷]HN | [SEQ ID NO:40] |
| [Ser¹, Arg⁷]HN-amide | [SEQ ID NO:40] |
| [Ser¹, Arg⁷]HN-(1-12) | [SEQ ID NO:411 |
| [Ser¹, Arg⁷]HN-(1-12)-amide | [SEQ ID NO:41] |
| [Ser¹, Arg⁷]HN-(1-10) | [SEQ ID NO:42] |
| [Ser¹, Arg⁷]HN-(1-10)-amide | [SEQ ID NO:42] |
| [Arg²]HN-(2-15) | [SEQ ID NO:43] |
| [Arg²]HN-(2-15)-amide | [SEQ ID NO:43] |
| [Arg²]HN-(2-12) | [SEQ ID NO:44] |
| [Arg²]HN-(2-12)-amide | [SEQ ID NO:44] |
| [Arg²]HN-(2-10) | [SEQ ID NO:45] |
| [Arg²]HN-(2-10)-amide | [SEQ ID NO:45] |
| [Arg⁷]HN-(2-15) | [SEQ ID NO:46] |
| [Arg⁷]HN-(2-15)-amide | [SEQ ID NO:46] |
| [Arg⁷]HN-(2-12) | [SEQ ID NO:47] |
| [Arg⁷]HN-(2-12)-amide | [SEQ ID NO:47] |
| [Arg⁷]HN-(2-10) | [SEQ ID NO:48] |
| [Arg⁷]HN-(2-10)-amide | [SEQ ID NO:48] |
| [Arg⁶]HN-(3-15) | [SEQ ID NO:49] |
| [Arg⁶]HN-(3-15)-amide | [SEQ ID NO:49] |
| [Arg⁶]HN-(3-12) | [SEQ ID NO:50] |
| [Arg⁶]HN-(3-12)-amide | [SEQ ID NO:50] |
| [Arg⁶]HN-(3-10) | [SEQ ID NO:51] |
| [Arg⁶]HN-(3-10)-amide | [SEQ ID NO:51] |
| [Arg⁶]HN-(4-15) | [SEQ ID NO:52] |
| [Arg⁶]HN-(4-15)-amide | [SEQ ID NO:52] |
| [Arg⁶]HN-(4-12) | [SEQ ID NO:53] |
| [Arg⁶]HN-(4-12)-amide | [SEQ ID NO:53] |
| [Arg⁶]HN-(4-10) | [SEQ ID NO:54] |
| [Arg⁶]HN-(4-10)-amide | [SEQ ID NO:54] |
| [Arg⁶]HN-(5-15) | [SEQ ID NO:55] |
| [Arg⁶]HN-(5-15)-amide | [SEQ ID NO:55] |
| [Arg⁶]HN-(5-12) | [SEQ ID NO:56] |
| [Arg⁶]HN-(5-12)-amide | [SEQ ID NO:56] |
| [Arg⁶]HN-(5-10) | [SEQ ID NO:57] |
| [Arg⁶]HN-(5-10)-amide | [SEQ ID NO:57] |
| [Arg⁶]HN-(6-15) | [SEQ ID NO:58] |
| [Arg⁶]HN-(6-15)-amide | [SEQ ID NO:58] |
| [Arg⁶]HN-(6-12) | [SEQ ID NO:59] |
| [Arg⁶]HN-(6-12)-amide | [SEQ ID NO:59] |
| [Arg⁶]HN-(6-10) | [SEQ ID NO:60] |
| [Arg⁶]HN-(6-10)-amide | [SEQ ID NO:60] |
| [Ser¹]HN-Gly¹⁶-Gly¹⁷ | [SEQ ID NO:61] |
| [Ser¹]HN-Gly¹⁶-Gly¹⁷-amide | [SEQ ID NO:61] |
| [Thr¹]HN-Gly¹⁶-Gly¹⁷ | [SEQ ID NO:62] |
| [Thr¹]HN-Gly¹⁶-Gly¹⁷-amide | [SEQ ID NO:62] |
| [Gly¹]HN-Gly¹⁶-Gly¹⁷ | [SEQ ID NO:63] |
| [Gly¹]HN-Gly¹⁶-Gly¹⁷-amide | [SEQ ID NO:63] |
| [Leu¹]HN-Gly¹⁶-Gly¹⁷ | [SEQ ID NO:64] |
| [Leu¹]HN-Gly¹⁶-Gly¹⁷-amide | [SEQ ID NO:64] |
| [A1a¹]HN-Gly¹⁶-Gly¹⁷ | [SEQ ID NO:65] |
| [Ala¹]HN-Gly¹⁶-Gly¹⁷-amide | [SEQ ID NO:65] |
| [Val¹]HN-Gly¹⁶-Gly¹⁷ | [SEQ ID NO:66] |
| [Val¹]HN-Gly¹⁶-Gly¹⁷-amide | [SEQ ID NO:66] |
| [Ile¹]HN-Gly¹⁶-Gly¹⁷ | [SEQ ID NO:67] |
| [Ile¹]HN-Gly¹⁶-Gly¹⁷-amide | [SEQ ID NO:67] |
| [Phe¹]HN-Gly¹⁶-Gly¹⁷ | [SEQ ID NO:68] |
| [Phe¹]HN-Gly¹⁶-Gly¹⁷-amide | [SEQ ID NO:68] |
| [Tyr¹]HN-Gly¹⁶-Gly¹⁷ | [SEQ ID NO:69] |
| [Tyr¹]HN-Gly¹⁶-Gly¹⁷-amide | [SEQ ID NO:69] |
| HN-Gly¹⁶-Gly¹⁷ | [SEQ ID NO:70] |
| HN-Gly¹⁶-Gly¹⁷-amide | [SEQ ID NO:70] |
| [Cys¹]HN-Gly¹⁶-Gly¹⁷ | [SEQ ID NO:71] |
| [Cys¹]HN-Gly¹⁶-Gly¹⁷-amide | [SEQ ID NO:71] |
| [Val¹, Val², Arg⁷]HN | [SEQ ID NO:72] |
| [Val¹, Val², Arg⁷]HN-amide | [SEQ ID NO:72] |
| [Lys¹]HN | [SEQ ID NO:73] |
| [Lys¹]HN-amide | [SEQ ID NO:73] |
| [Lys¹]HN-(1-12) | [SEQ ID NO:74] |
| [Lys¹]HN-(1-12)-amide | [SEQ ID NO:74] |
| [Lys¹]HN-(1-10) | [SEQ ID NO:75] |
| [Lys¹]HN-(1-10)-amide | [SEQ ID NO:75] |
| [Ser¹, Lys²]HN | [SEQ ID NO:76] |
| [Ser¹, Lys²]HN-amide | [SEQ ID NO:76] |
| [Ser¹, Lys²]HN-(1-12) | [SEQ ID NO:77] |
| [Ser¹, Lys²]HN-(1-12)-amide | [SEQ ID NO:77] |
| [Ser¹, Lys²]HN-(1-10) | [SEQ ID NO:78] |
| [Ser¹, Lys²]HN-(1-10)-amide | [SEQ ID NO:78] |
| [Ser¹, Lys⁷]HN | [SEQ ID NO:79] |
| [Ser¹, Lys⁷]HN-amide | [SEQ ID NO:79] |
| [Ser¹, Lys⁷]HN-(1-12) | [SEQ ID NO:80] |
| [Ser¹, Lys⁷]HN-(1-12)-amide | [SEQ ID NO:80] |
| [Ser¹, Lys⁷]HN-(1-10) | [SEQ ID NO:81] |
| [Ser¹, Lys⁷]HN-(1-10)-amide | [SEQ ID NO:81] |
| [Lys²]HN-(2-15) | [SEQ ID NO:82] |
| [Lys²]HN-(2-15)-amide | [SEQ ID NO:82] |
| [Lys²]HN-(2-12) | [SEQ ID NO:83] |
| [Lys²]HN-(2-12)-amide | [SEQ ID NO:83] |
| [Lvs²]HN-(2-10) | [SEQ ID NO:84] |
| [Lys²]HN-(2-10)-amide | [SEQ ID NO:84] |
| [Lys⁷]HN-(2-15) | [SEQ ID NO:85] |
| [Lys⁷]HN-(2-15)-amide | [SEQ ID NO:85] |
| [Lys⁷]HN-(2-12) | [SEQ ID NO:86] |
| [Lys⁷]HN-(2-12)-amide | [SEQ ID NO:86] |
| [Lys⁷]HN-(2-10) | [SEQ ID NO:87] |
| [Lys⁷]HN-(2-10)-amide | [SEQ ID NO:87] |
| HN-(3-15) | [SEQ ID NO:88] |
| HN-(3-15)-amide | [SEQ ID NO:88] |
| HN-(3-12) | [SEQ ID NO:89] |
| HN-(3-12)-amide | [SEQ ID NO:89] |
| HN-(3-10) | [SEQ ID NO:90] |
| HN-(3-10)-amide | [SEQ ID NO:90] |
| HN-(4-15) | [SEQ ID NO:91] |
| HN-(4-15)-amide | [SEQ ID NO:91] |
| HN-(4-12) | [SEQ ID NO:92] |
| HN-(4-12)-amide | [SEQ ID NO:92] |
| HN-(4-10) | [SEQ ID NO:93] |
| HN-(4-10)-amide | [SEQ ID NO:93] |
| HN-(5-15) | [SEQ ID NO:94] |
| HN-(5-15)-amide | [SEQ ID NO:94] |
| HN-(5-12) | [SEQ ID NO:95] |
| HN-(5-12)-amide | [SEQ ID NO:95] |
| HN-(5-10) | [SEQ ID NO:96] |
| HN-(5-10)-amide | [SEQ ID NO:96] |
| HN-(6-15) | [SEQ ID NO:97] |
| HN-(6-15)-amide | [SEQ ID NO:97] |
| HN-(6-12) | [SEQ ID NO:98] |
| HN-(6-12)-amide | [SEQ ID NO:98] |
| HN-(6-10) | [SEQ ID NO:99] |
| HN-(6-10)-amide | [SEQ ID NO:99] |
| [Ser¹]HN-Cys¹⁶ | [SEQ ID NO:100] |
| [Ser¹]HN-Cys¹⁶-amide | [SEQ ID NO:100] |
| [Thr¹]HN-Cys¹⁶ | [SEQ ID NO:101] |
| [Thr¹]HN-Cys¹⁶-amide | [SEQ ID NO:101] |
| [Gly¹]HN-Cys¹⁶ | [SEQ ID NO:102] |
| [Gly¹]HN-Cys¹⁶-amide | [SEQ ID NO:102] |
| [Leu¹]HN-Cys¹⁶ | [SEQ ID NO:103] |
| [Leu¹]HN-Cys¹⁶-amide | [SEQ ID NO:103] |
| [Ala¹]HN-Cys¹⁶ | [SEQ ID NO:104] |
| [Ala¹]HN-Cys¹⁶-amide | [SEQ ID NO:104] |
| [Val¹]HN-Cys¹⁶ | [SEQ ID NO:105] |
| [Val¹]HN-Cys¹⁶-amide | [SEQ ID NO:105] |
| [Ile¹]HN-Cys¹⁶ | [SEQ ID NO:106] |
| [Ile¹]HN-Cys¹⁶-amide | [SEQ ID NO:106] |
| [Phe¹⁶]HN-Cys¹⁶ | [SEQ ID NO:107] |
| [Phe¹]HN-Cys¹⁶-amide | [SEQ ID NO:107] |
| [Tyr¹]HN-Cys¹⁶ | [SEQ ID NO:108] |
| [Tyr¹]HN-Cys¹⁶-amide | [SEQ ID NO:108] |
| HN-Cys¹⁶ | [SEQ ID NO:109] |
| HN-Cys¹⁶-amide | [SEQ ID NO:109] |
| [Cys¹]HN-Cys¹⁶ | [SEQ ID NO:110] |
| [Cys¹]HN-Cys¹⁶-amide | [SEQ ID NO:110] |
| [Val¹, Val², Lys⁷]HN | [SEQ ID NO:111] |
| [Val¹, Val², Lys⁷]HN-amide | [SEQ ID NO:111] |
| HN-(2-15) | [SEQ ID NO:112] |
| HN-(2-15)-amide | [SEQ ID NO:112] |
| HN-(7-15) | [SEQ ID NO:113] |
| HN-(7-15)-amide | [SEQ ID NO:113] |
| HN-(1-15) | [SEQ ID NO:114] |
| HN-(1-15)-amide | [SEQ ID NO:114] |
| [Ser¹]HN-(1-14) | [SEQ ID NO:115] |
| [Ser¹]HN-(1-14)-amide | [SEQ ID NO:115] |
| [Ser¹]HN-(1-13) | [SEQ ID NO:116] |
| [Ser¹]HN-(1-13)-amide | [SEQ ID NO:116] |
| [Ser¹, His²]HN | [SEQ ID NO:117] |
| [Ser¹, His²]HN-amide | [SEQ ID NO:117] |
| [Gln¹]HN | [SEQ ID NO:118] |
| [Gln¹]HN-amide | [SEQ ID NO:118] |
| [Ser¹, Ala²]HN | [SEQ ID NO:119] |
| [Ser¹, Ala²]HN-amide | [SEQ ID NO:119] |
| [Ser¹, Ser²]HN | [SEQ ID NO:120] |
| [Ser¹, Ser²]HN-amide | [SEQ ID NO:120] |
| [Glu¹]HN | [SEQ ID NO:121] |
| [Glu¹]HN-amide | [SEQ ID NO:121] |
| [Ser¹]HN-Gly¹⁶ | [SEQ ID NO:122] |
| [Ser¹]HN-Gly¹⁶-amide | [SEQ ID NO:122] |
| [Ser¹, His]HN | [SEQ ID NO:123] |
| [Ser¹, His²]HN-amide | [SEQ ID NO:123] |
| HN-(2-10) | [SEQ ID NO:124] |
| HN-(2-10)-amide | [SEQ ID NO:124] |
| [Arg²]HN | [SEQ ID NO:125] |
| [Arg²]HN-amide | [SEQ ID NO:125] |
| [Lys²]HN | [SEQ ID NO:126] |
| [Lys²]HN-amide | [SEQ ID NO:126] |
| [pGlu¹]HN | [SEQ ID NO:127] |
| [pGlu¹]HN-amide | [SEQ ID NO:127] |
| [Val¹, Val², Arg⁷, (3nitro-2- | [SEQ ID NO:128] |
| sulfenyl-pyridine)Cys¹⁰]HN | |
| [Val¹, Val², Thr⁴, Arg⁷]HN | [SEQ ID NO: 129] |

In a further embodiment of the present invention the Histogranin compounds are cyclic peptides including compounds which have been defined in Canadian Patent Applications 2,306,754 and 2,475,609, both of which are incorporated herein by reference. The method of preparing said cyclic compounds are also provided in the afore mentioned Canadian Patent Applications.

Examples of cyclic peptides and pseudopeptides have the following Formula I (see U.S. Pat. No. 6,566,327 or Canadian Patent Application No. 2,306,754):

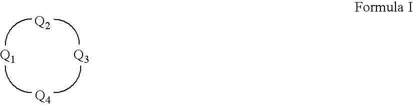

wherein
Q₁ represents glycine, alanine, valine, leucine, isoleucine, lysine, histidine or arginine;
Q₂ represents asparagine or glutamine,
Q₃ represents glycine, alanine, valine, leucine, isoleucine, phenylalamine, tryptophan, or tyrosine: and
Q₄ represents lysine, arginine, or histidine;
pseudopeptide analogues thereof wherein one or more of the carbonyl groups of the peptide linkage is replaced by —C(═S)— or by —CH₂—, and/or wherein one or more of the amide bonds, —C(O)—NH—, is replaced by the retro-verso form, —NH—C(O)—, thereof; and
pharmaceutically acceptable salts and esters thereof.

More preferred peptides and pseudopeptides are represented by Formula I wherein

Q₁ represents glycine or arginine:
Q₂ represents L-glutamine or D-glutamine;
Q₃ represents glycine, alanine, or tyrosine;
Q₄ represents L-arginine or D-arginine.

Specific compounds of the invention include the following:

| | |
|---|---|
| H-Arg-Gln-Gly-Arg-OH | (SEQ ID NO:130) |
| H-Gly-Gln-Gly-Arg-OH | (SEQ ID NO:131) |
| H-Gly-Gln-Ala-Arg-OH | (SEQ ID NO:132) |
| H-Arg-Gln-Ala-Arg-OH | (SEQ ID NO:133) |
| cyclic (-Gly-Gln-Ala-Arg-) | |
| cyclic (-Gly-Gln-Ala-Arg-Gly-Gln- | |
| Ala-Arg-) | |
| cyclic (-Arg-Gln-Ala-Arg-) | |
| cyclic (-Arg-G1n-Ala-Arg-Arg-Gln- | |
| Ala-Arg-) | |
| cyclic (-Gly-Gln-Tyr-Arg-) | |
| cyclic (-Gly-Gln-Tyr-D-Arg-) | |
| cyclic (-Gly-D-Gln-Tyr-D-Arg-) | |
| H-Gly-Gln-Tyr-Arg-OH | (SEQ ID NO:134) |
| H-Gly-Gln-Tyr-D-Arg-OH | (SEQ ID NO:135) |
| H-Gly-D-Gln-Tyr-D-Arg-OH | (SEQ ID NO:136) |
| H-Arg-Gln-G1y-Arg-Thr-Leu-Tyr-Gly- | (SEQ ID NO:137) |
| Phe-OH | |

Cyclic polypeptides are also represented by the following general Formula II (see US Publication No. 20030176329 or Canadian Patent Application No. 2,475,609):

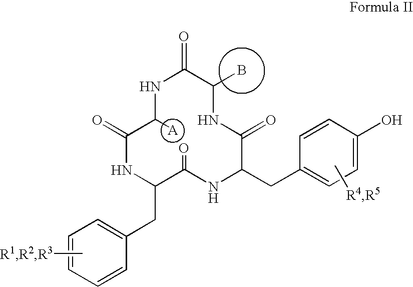

wherein:

A is -hydrogen, —(C₁-C₈) alkyl or —(C₁-C₈) alkyl substituted by hydroxy;

B is —(C₁-C₆) alkylguanidino, —(C₁-C₆) alkyl (4-imidazolyl), —(C₁-C₆) alkylamino, p-aminophenylalkyl (C₁-C₆)—, p-guanidinophenylalkyl (C₁-C₆)— or 4-pyridinylalkyl (C₁-C₆)—;

R¹, R² and R³ are, independent of one another, -hydrogen, -arylcarbonylamino, —(C₁-C₆) alkoylamino, —(C₁-C₆) alkylamino, —(C₁-C₆) alkyloxy, —(C₁-C₆) alkylaminocarbonyl, -carboxy, —OH, -benzoyl, -p-halogenobenzoyl, -methyl, —S-(2,4-dinitrophenyl), —S-(3-nitro-2-pyridinesulfenyl), -sulfonyl, -trifluoromethyl, —(C₁-C₆) alkylaminocarbonylamino, -halo or -amino;

R⁴ and R⁵ are, independent of one another, -hydrogen, —(C₁-C₆)alkyl, -methyloxy, -nitro, -amino, -arylcarbonylamino, —(C₁-C₆) alkoylamino, —(C₁-C₆) alkylamino, -halo or —OH.

Examples of cyclic peptides of Formula II include for example:

### Cyclic (-Gly-(p-chloro) Phe-Tyr-D-Arg-) [Compound II-1)

### Cyclic (-Gly-(p-chloro) Phe-Tyr-(p-amino)Phe-) [Compound II-2]

### Cyclic (-Gly-(p-chloro) Phe-Tyr-(p-guanidino)Phe-) [Compound II-3]

### Cyclic (-Gly-(p-amino) Phe-Tyr-D-Arg-) [Compound II-4)

### Cyclic (-Thr-(p-chloro) Phe-Tyr-D-Arg-) [Compound II-5]

Non-peptides based on the histogranin peptide are also included in the present invention as defined by Formula III and IV below (see US Publication No. 2003016329 or Canadian Patent Application No. 2,475,609):

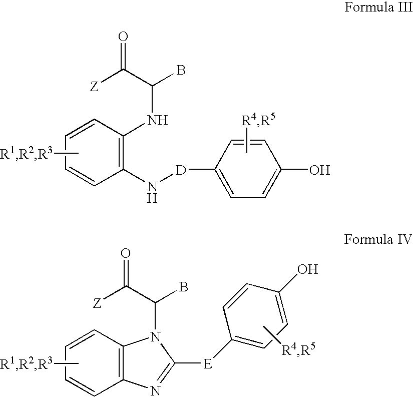

wherein:

B is —(C₁-C₆) alkylguanidino, —(C₁-C₆) alkyl (4-imidazolyl), —(C₁-C₆) alkylamino,

p-aminophenylalkyl (C₁-C₆)—, p-guanidinophenylalkyl (C₁-C₆)— or 4-pyridinylalkyl (C₁-C₆)—;

D is —(CO)—, —(CO)— (C₁-C₆) alkylene or —(C₁-C₆) alkylene;

E is a single bond or —(C₁-C₆) alkylene;

Z is —NH₂, —NH— (C₁-C₆) alkylcarboxamide, —NH— (C₁-C₆) alkyl, —NH-benzyl, —NH-cyclic (C₁-C₇) alkyl, —NH-2-(1-piperidyl)ethyl, —NH-2-(1-pyrrolidyl)ethyl, —NH-2-(1-pyridyl)ethyl, —NH-2-(morpholino) ethyl, -morpholino, -piperidyl, —OH, —(C₁-C₆) alkoxy, —O-benzyl or —O-halobenzyl;

R¹, R² and R³ are, independent of one another, -hydrogen, -arylcarbonylamino, —(C₁-C₆) alkoylamino, —(C₁-C₆) alkylamino, —(C₁-C₆) alkyloxy, —(C₁-C₆) alkylaminocarbonyl, -carboxy, —OH, -benzoyl, -p-halogenobenzoyl, -methyl, —S-(2,4-dinitrophenyl), —S-(3-nitro-2-pyridinesulfenyl), -sulfonyl, -trifluoromethyl, —(C₁-C₆) alkylaminocarbonylamino, -halo or -amino;

R⁴ and R⁵ are, independent of one another, -hydrogen, —(C₁-C₆)alkyl, -methyloxy,

-nitro, -amino, -arylcarbonylamino, —(C₁-C₆) alkoylamino, —(C₁-C₆) alkylamino, -halo or —OH.

Examples of non-peptides of Formula III are found below:
N-5-guanidinopentanamide-(2S)-yl -2-N (p-hydroxyphenylacetyl)phenylenediamine [Compound III-1]
N-5-guanidinopentanamide-(2S)-yl-2-N-(p-hydroxyphenylacetyl)-4-trifluoromethyl-phenylenediamine [Compound III-2]
N-5-guanidinopentanamide-(2R)-yl -2-N-(p-hydroxyphenylacetyl) 4-carboxy-phenylenediamine [Compound III-3]
N-5-guanidinopentanamide-(2R)-yl-2-N-(p-hydroxyphenylacetyl) 4-(p-chlorobenzoyl)-phenylenediamine [Compound III-4]
N-5-guanidinopentanamide-(2R)-yl-2-N-(p-hydroxyphenylacetyl)-4-phenylenediamine [Compound III-5]

An example of a non-peptide of Formula IV includes for example:
N-5-guanidinopentanamide-(2R)-yl -2-(p-hydroxybenzyl)-5-carboxybenzimidazole [Compound IV-1]

In yet a further embodiment of the present invention, additional useful compounds include the cyclic peptides represented by the general formula V, as shown below.

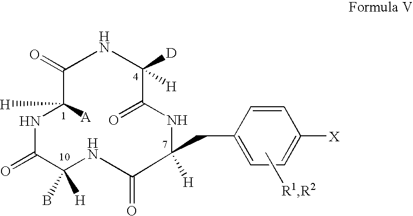

wherein:
Carbon atoms in positions 1, 4, 7 and 10 can be under the configurations S or R, but preferably S, S, S and R, respectively.
“A” is hydrogen, —(C₁-C₈)alkyl, —(C₁-C₈)alkyl substituted by hydroxyl or —(C₁-C₈)alkyl substituted by sulfur;
“B” is —(C₁-C₆)alkylguanidino, —(C₁-C₆)alkyl(4-imidazolyl) or (C₁-C₆)alkylamino;
“D” is H, methyl, —(C₁-C₈)alkyl, —(C₁-C₈)alkyl substituted by hydroxyl or —(C₁-C₈)alkyl substituted by sulphur;
R¹ and R² are, independent of one another, hydrogen, —(C₁-C₆)alkyl, methyloxy, nitro, amino, arylcarbonylamino, (C₁-C₆)alkoylamino, (C₁-C₆)alkylamino, halo or hydroxy.
“X” is hydrogen, hydroxyl or halogen.

An example of a cyclic peptide of Formula V includes for example: cyclic-Gly-Ala-Tyr-D-Arg.

In some embodiments of the present invention the peptides that act as inhibitors of P2X₇ receptor function include the peptides and non-peptides shown in Table 1.

TABLE 1

| COMPOUND | NAME/STRUCTURE | REFERENCE |
|---|---|---|
| 1 | Histogranin (HN) | Eur J. Pharmocol. |
| | H-Met-Asn-Tyr-Ala-Leu-Lys-Gly- | 1993 |
| | Gln-Gly-Arg-Thr-Leu-Tyr-Gly-Phe- | 245:247-56 |
| | COOH | |
| 2 | [Ser^{1]}HN | Canadian Patent |
| | H-Ser-Asn-Tyr-Ala-Leu-Lys-Gly- | Application |
| | Gln-Gly-Arg-Thr-Leu-Tyr-Gly-Phe- | 2,097,533 |
| | COOH | |
| 3 | HN-(7-15) | Canadian Patent |
| | H-Gly-Gln-Gly-Arg-Thr-Leu-Tyr- | Application |
| | Gly-Phe-COOH | 2,097,533 |
| 4 | [Val¹, Val², Arg⁷]HN or H4- | Canadian Patent |
| | (86-100) | Application |
| | Val-Val-Tyr-Ala-Leu-Lys-Arg-Gln- | 2,097,533 |
| | Gly-Arg-Thr-Leu-Tyr-Gly-Phe- | |
| 5 | [Val¹, Val², Arg⁷, [S-(3nitro-2- | |
| | pyridine sulfenyl)]Cys¹⁰]HN | |
| | H-Val-Val-Tyr-Ala-Leu-Lys-Arg- | |
| | Gln-Gly-[S-(3nitro-2-pyridine | |
| | sulfenyl)]Cys-Thr-Leu-Tyr-Gly-Phe- | |
| 6 | [Val¹. Val².Thr⁴.Arg⁷]HN | |
| | Val-Val-Tyr-Thr-Leu-Lys-Arg-Gln- | |
| | Gly-Arg-Thr-Leu-Tyr-Gly-Phe | |

Cyclic peptides

| | | |
|---|---|---|
| 7 | Peptide 8 | Canadian Patent |
| | Cyclic (-Gly-Gln-Tyr-D-Arg-) | Application |
| | | 2,306,754; |
| | | US Pat. No. |
| | | 6,566,397 |
| 8 | Peptide 9 | Canadian Patent |
| | Cyclic (-Gly-D-Gln-Tyr-D-Arg-) | Application |
| | | 2,306,754; |
| | | US Pat. No. |
| | | 6,566,327 |
| 9 | Compound V-1 | |
| | Cyclic [Ala⁸, Tyr⁹, D-Arg¹⁰] | |
| | HN-7-l0) | |
| | cyclic (-Gly-Ala-Tyr-D-Arg-) | |
| 10 | Compound II-1 | Canadian Patent |
| | Cyclic (-Gly-(p-chloro)Phe-Tyr-D- | Application |
| | Arg-) | 2,475.609; |
| | | U.S. patent |
| | | application |
| | | 20030176329 |

Non-peptides

| | | |
|---|---|---|
| 12 | Compound III-5 | Canadian Patent |
| | N-5-guanidinopentanamide-(2R)-yl- | Application |
| | 2-N-(p-hydroxphenylacetyl) | 2.475,609; |
| | phenylenediamine | U.S. patent |
| | | application |
| | | 20030176329 |
| 13 | Compound III-4 | Canadian Patent |
| | N-5-guanidinopentanamide-(2R)-yl- | Application |
| | 2-N-(p-hydroxyphenylacetyl)-4-(p- | 2,475,609; |
| | chlorobenzoyl)-phenylenediamine | U.S. patent |
| | | application |
| | | 20030176329 |
| 15 | Compound IV-1 | Canadian Patent |
| | N-5-guanidinopentanamide-(2R)-yl | Application |
| | 2-(p-hydroxylbenzyl)5- | 2,475,609; |
| | carboxybenzimidazole | U.S. patent |
| | | application |
| | | 20030176329 |

In one aspect, the present invention relates to the use of Histogranin compounds for reducing P2X₇ receptor-like activities in animal cells. The invention encompasses the use of any animal cells including human cells. These cells may be in the form of primary cell preparations or immortalized cell lines including cells transfected with a gene. These cells can be isolated from tissue or organs using techniques known to those of skill in the art. For example, immune cells can be collected or isolated from blood or secondary lymphoid organs of the subject such as, but not limited to, lymph nodes, tonsils, spleen. Peyer's patch of the intestine, and bone marrow, by any of the methods known in the art (see. e.g. Current Protocols in Immunology (1991) Green Publishing Associates, New York, N.Y. p. 21). Immune cells obtained from such sources typically comprise predominantly recirculating lymphocytes and macrophages at various stages of differentiation and maturation. The immune cells used in the in vitro methods of the invention can be collected by standard techniques, such as by use of a syringe to withdraw the blood, followed by subjecting the blood to Ficoll-Hypaque (Pharmacia) gradient centrifugation. Monocytes-derived macrophages can be isolated from immune cells by allowing the cells to adhere to culture flasks for 1-2 hours, washing away non-adherent cells and culturing adherent cells for 7-12 days in medium (i.e. RPMI 1640 supplemented with 20% human serum. 2 mM glutamine. 5 mM HEPES and 100 mμ.g/ml streptomycin (see, e.g. Blanchard et al. (1995) J. Cell Biochem. 57:452-464; Blanchard et al. (1994) J. Immunol. 147:2579-2585). Alveolar macrophages can be obtained by bronchoalveolar lavage as described in the example below and in Lemaire et al. (1985) Am. Rev. Respir. Dis. 131:144-149; Lemaire (1991) Am. J. Pathol. 138:487-495; Lemaire et al. (1996) J. Immunol. 157:5118-5125). In one embodiment of the present invention, UN compounds reduce ATP and BzATP-induced pore formation in primary cultures of rat alveolar macrophages (AM), a cell preparation shown to express a functional P2X₇ receptor (Lemaire et al. (2003) Drug Dev. Res. 59:118-127). This activity is a hallmark of the P2X₇ receptor and has been used successfully as a reliable measure of P2X₇ receptor activation (Steinberg et al. (1987) J. Biol. Chem. 262:8884-8888; rev. in Di Virgilio et al. (2001) Blood 97:587-600; Falzoni et al. (1995) J. Clin. Invest. 95:1207-1216; Raouf et al. (2004) Mol. Pharmacol. 65:646-654; Donnelly-Roberts et al. (2004) J. Pharmacol. Exp. Ther. 308:1053-1061; Lundy et al. (2004) Eur. J. Pharmacol. 487:17-28; Alcaraz et al. (2003) Biorg. Med. Chem. Lett. 13:4043-4046). This was determined by measuring the entry of the fluorescent DNA probe, ethidium bromide (EtBr) through the membrane pores with a consequent increase in cell nuclear fluorescence. Inhibition of ATP-pore forming activity used to test the subject of the present invention has been previously used and validated for the assessment of P2X₇ receptor inhibition (Alcaraz et al. (2003) Biorg. Med. Chem. Lett. 13:4043-4046). This could be performed by monitoring the fluorescence levels in a cell suspension as a function of time or at a given time interval by spectrophotometry (Falzoni et al. (1995) J. Clin. Invest. 95:1207-1215; Lundy et al. (2004) Eur. J. Pharmacol. 487:17-28) or flow cytometry (FACS) analysis (Nihei et al. (2000) Mem. Inst. Oswaldo Cruz. Rio de Janeiro, 95:415-428; Sanders et al. (2003) J. Immunol. 171:5442-5446), or microscopic examination and capture image analysis (Lammas et al. (1997) Immunity 7:433-444; Fairbairn et al. (2001) J. Immunol. 167:3300-3307; Coutinho-Silva et al. (2001) Am. J. Cell Physiol. 280:C81-C89; Lemaire et al. (2003) Drug Dev. Res. 59:118-127; and as in example below). Any variety of fluorescent dyes may be used to assess HN compounds reduction of P2X₇ receptor-mediated pore formation of the present invention. Examples of such probes include but are not limited to Lucifer yellow (Steinberg et al. (1987) J. Biol. Chem. 262:3118-3122) and YO-PRO (Virginio et al. (1999) J. Physiol. 519:335-346). Any agonist at the P2X₇ receptor may be used to test the subject of the present invention.

In other embodiments of the present invention, HN compounds reduce ATP-induced apoptosis and cell death in rat AM. A striking feature of P2X₇ receptor activation following longer exposure to ATP is the induction of a process of cell death sharing apoptosis and necrosis termed “aponecrosis” (Formegli et al. (2000) J. Cell Physiol. 182:41-49). ATP-induced apoptosis and cell death has been used as a measure of P2X₇ receptor activity (Ferrari et al. (1997) Neuropharmacology 36:1295-1301; Schulze-Lohoff et al. (1998) Am. J. Physiol. 275 (Renal Physiol. 44):F962-F971; Brough et al. (2002) Mol. Cell. Neurosci. 19:272-280; Ferrari et al. (1999) FEBS Lett. 447:71-75). Methods of measuring the P2X₇ receptor-induced apoptosis are described herein in the example below and in the literature. Examples of such methods include but are not limited to: ATP-induced changes in cell morphology characteristic of various stages of apoptosis such as nuclear chromatin condensation detected by microscopic evaluation of increased nuclei fluorescence using Hoechst-33342 stain (Ferrari et al. (1999) FEBS Lett. 447:71-75 and the example disclosed herein); ATP-induced histone-associated DNA fragments, a measure of internucleosomal degradation of genomic DNA occurring during apoptosis, detected by specific cell death ELISA (Boehringer Mamheim) (Li et al. (1997) Toxicol. Appl. Pharmacol. 145:331-339; Lemaire et al. (2003) Drug Dev. Res. 59:118-127; and in the example disclosed herein); and ATP-induced DNA fragmentation analysed by DNA isolation and DNA ladder gel electrophoresis (Lemaire et al. (2003) Drug Dev. Res. 59:118-127). P2X₇-induced cell death can be assessed by measurement of secondary necrosis detected by uptake of propidium iodide (Elliott et al. (2005) Arthritis Res. Ther. 7:R468-R475) and/or by trypan blue uptake and release of the cytoplasmic enzyme lactate dehydrogenase (LDH) (Ferrari et al. (1999) FEBS Lett. 447:71-75), or ⁵¹Chromium in the culture medium (Li et al. (2002) FEBS Lett. 531:127-131).

In another embodiment of the present invention. HN compounds reduce P2X₇-induced release of IL-1.beta and IL-1.alpha in rat alveolar macrophages. ATP-induced posttranslational processing and release of IL-1.beta and IL-1.alpha represent a selective measure of P2X₇-mediated activity (Solle et al. (2001) J. Biol. Chem. 276:125-132). Techniques known to those with skill in the art can be used to measure the inhibition of P2X₇-dependent IL-1 production in animal cells. For example, following incubation with LPS, cells are briefly stimulated with ATP and changes in ATP-induced posttranslational processing and production of interleukins can be measured using immunoprecipitation and/or ELISA assays (Perregaux et al. (1994) J. Biol. Chem. 269:15195-15203; Perregaux et al. (1998) J. Immunol. 160:2469-2477; Ferrari et al. (1997) J. Immunol. 159:1451-1458; e.g. as further described in the example below).

In other embodiments, HN compounds inhibit in rat macrophages, rapid ATP-induced phosphatidylserine (PS) translocation from the inner leaflet of the plasma membrane to the outer, a process referred to as “PS flip”. PS flip following brief (≦10 min.) P2X₇ receptor activation is reversible, is not associated with cell death and precedes membrane shedding of microvesicles that contain fully processed IL-1.beta (Mackenzie et al. (2001) Immunity 8:825-835). This is thought to be the major secretory pathway for rapid release of this cytokine. Moreover, following longer exposure to ATP and P2X₇ receptor activation, PE is exposed irreversibly at the cell surface and PS and associated proteins are major targets of autoantibodies in systemic lupus erythematosus (McClain et al. (2004) Arthritis Rheum. 50:1226-1932). The methods used for assessing the inhibition of HN compounds on PS flip associated with microvesicle shedding can be found in the art: for example, cells expressing the P2X₇ receptor are incubated with fluorescently labeled annexin-V, a high affinity PS binding protein (Andree et al. (1990) J. Biol. Chem. 265:4923-4928). After a very brief (1-5 min) stimulation with ATP or BzATP or any P2X₇ agonist, fluorescent annexin-V specifically bind to the exposed PS on the outer surface of cells. Typically, prior to agonist application, smaller than 2% of cells are annexin-positive while >95% of cells become annexin-positive following agonist stimulation. Any variety of fluorescent probe (ex. fluorescein as in example below or rhodamine) can be conjugated to annexin-V; cell membrane fluorescence can be measured as a function of time or at a given time interval by digital video microscopy or capture image analysis (Mackenzie et al. (2001) Immunity 8:825-835; as described in example below) or by flow cytometry (Hammill et al. (1999) Exp. Cell Res. 251:16-21; Satta et al. (1994) J. Immunol. 153:3945-3255: Dachary-Prigent et al. (1993) Blood 81:2554-2565).

This invention provides, in another aspect a method for reducing P2X₇ receptor-stimulated IL-1.beta, IL-1.alpha, IL-18 and IL-6 release in mammals. Several studies have demonstrated that ATP acting at the P2X₇ receptor is required as a second stimulus to generate the extracellular release of these cytokines (Perregaux et al. (1994) J. Biol. Chem. 269:15195-15203; Sanz et al. (2000) J. Immunol. 164:4893-4898; Griffiths et al. (1995) J. Immunol. 154:2821-2828; Solle et al. (2001) J. Biol. Chem. 276:125-132; Perregaux et al. (2000) J. Immunol. 165:4615-4623; Mehta et al. (2001) J. Biol. Chem. 276:3820-3826; Sluyter et al. (2004) Genes Immun. 5:585-591). Thus an initial i.p. injection of mice with LPS generates little release of extracellular cytokines. Subsequent i.p. injection of ATP results in the extracellular release of large quantities of IL-1.beta and IL-1.alpha (Griffiths et al. (1995) J. Immunol. 154:2821-2828). The method of the present invention comprises injecting i.p. RN compounds under conditions (i.e. prior to or concomitantly to ATP) such that extracellular release of cytokines is reduced. Following treatment of mice, lavage of the peritoneal cavity can be performed and peritoneal lavage fluid can be collected by techniques known to those of skill in the art (Griffiths et al. (1995) J. Immunol. 154:2821-2828; and as described in example below). IL-1.beta, IL-1.alpha and IL-6 protein in peritoneal lavage fluids can be measured by immunoprecipitation and/or ELISA assays (e.g. as further described in the example below).

In another application, HN compounds can be used to reduce the symptoms that develop in certain models of disease where the P2X₇ receptor may mediate cell death, tissue injury and the release of IL-1.beta, such as inflammatory and autoimmune diseases and neurodegenerative diseases. For example, if a symptom of inflammation or neurodegeneration is reduced or absent in a mammal or animal cell under conditions that induce such a symptom in a corresponding non-treated mammals or animal cell then the HN compounds play a role in curbing the symptoms. Inflammation can be assessed in animals including but not restricted to, mice treated or not with HN compounds following the onset of inflammation disease. Examples of these included but are not restricted to: osteoarthritis induced e.g. by anterior cruciate ligament transection (ACLT) (Tiraloche et al. (2005) Arthritis Rheum. Apr. 52(4): 1118-1128) or by surgical transection of the medial meniscotibial ligament (Glasson et al. (2005) Nature Mar. 31; 434(7033):644-648) or by intra-articular injection of iodoacetate (Pomonis et al. (2005) Pain Apr. 114 (3):339-346) or by administration of oral or parenteral quinolone antibiotics (rev. in Bendele (2001) J. Musculo-Skelet. Neuronal Interact., June; 1(4):363-376); asthma induced e.g. by ovalbumin sensitization using the Brown Norway rat model or by administering Sephadex (Belvisi et al. (2005) J. Pharmacol. Exp. Ther. May 5) or by challenge with hapten, homologous protein conjugate of the hapten or antigen (as reviewed in Paululun et al. (2005) Exp. Toxicol. Pathol. 56:203-234); inflammatory bowel disease (IBD) such as ulcerative colitis and Crohn's disease induced by administering dextran sodium sulfate (Berberat et al. (2005) Inflamm. Bowel Dis. 11:350-359) or by administering trinitrobenzene sulfonic acid (Aharoni et al. (2005) Inflamm. Bowel Dis. 11:106-115) or using methods as reviewed in Powrie et al. (2004) Novartis Found. Symp. 263:164-174); atherosclerosis induced in apolipoprotein E heterozygote mice by bacterial exposure (Chi et al. Circulation (2004) 110:1678-1685) or apolipoprotein E knock-out mice (Yeganch et al. (2005) J. Nutr. Biochem. 16:222-228); systemic lupus erythematosus using the New Zealand Black and New Zealand White (NZBXNZW) F₁ hybrid mouse model of SLE (Elliott J. et al. (2005) Arthritis Res. 7:R468-R475; Drake et al. (1994) Proc. Natl. Acad. Sci. (USA) 91:4062-4066; Kono et al. (1994) Proc. Natl. Acad. Sci. (USA) 91:10168-10172). The inflammation can be detected histologically by computed tomography (CT), magnetic resonance imaging (MRI), ultrasonography, scintigraphic imaging or microscopic observations of symptoms such as inflammatory cell infiltrates or tissue lesions.

HN compounds can be used to reduce symptoms of neurodegenerative diseases in models of neurodegeneration. For example. Alzheimer's disease can be induced in a mouse, by expressing human familial Alzheimer's disease (FAD) beta-amyloid precursor (APP) in the mouse, overexpressing human mild-type APP in mouse, overexpressing beta-amyloid 1-42 (beta.A) in the mouse, or expressing FAD presenillin-1 (PS-1) in the mouse (see. e.g. Higgins (1999) Mol. Med. Today 5:274-276). Gene products with altered concentrations in mice with Alzheimer's disease which can be measured include, but are not limited to amyloid.beta-peptides (A.beta.), tau protein, and neuronal thread protein (NTP) in the CSF of the mouse. Stroke can be induced in mouse, for example, by middle carotid artery occlusion (see, e.g., Garcio et al. (1995) Am. J. Pathol. 147:1477-1486; Hara et al. (1997) Proc. Natl. Acad. Sci. (USA) 94:2007-2012).

In yet another aspect, the invention provides a new method for the treatment of rheumatoid arthritis (RA). In this method HN compounds are injected i.p. before or after the onset of arthritis. Treatment in vivo with HN compounds reduces the symptoms associated with the development of collagen-induced arthritis (CIA) in mice. The mouse CIA model used to test UN compounds has been the model of choice in terms of testing potential new therapeutic agents for the treatment of human RA (Takaoka et al. (1998) Gen. Pharmac. 30:777-780; Wallace et al. (1999) J. Immunol. 162:5547-5555; Labasi et al. (2002) J. Immunol. 168:6436-6445; Malfait et al. Aug. 15, 2000 Proc. Natl. Acad. Sci. (USA); 97:9561-9566; Kim et al. (2002) Arthritis Rheum. 46:793-801; Joosten et al. (1996) Arthritis Rheum. 39:797-809) since the histopathology of inflammatory arthritis resembles human rheumatoid arthritis (Luross et al. (2001) Immunology 103:407-416). The onset of arthritis can be induced e.g. by administering collagen or collagen fragments (Joosten et al. (1999) J. Immunol. 163:5049-5055; Trentham et al. (1977) J. Exp. Med. 146:857-868; Campbell et al. (2001) J. Clin. Invest. 107:1519-1527: as described in example below); by administering collagen antibodies (Stuart et al. (1982) J. Exp. Med. 155:1-16; Watson et al. (1987) Arthritis Rheum. 30:460-465) or collagen monoclonal antibodies (Terato et al. (1992) J. Immunol. 148:2103-2108; Myers et al. (1995) 1 mmol. 84:509-513). Bacterial toxins (for example, LPS) can be used to trigger and enhance classic CIA (Yoshino et al. (2000) J. Immunol. 163:3417-3422; as described in example below; Cole et al. (1993) Arthritis Rheum. 36:994-1002; Takaoka et al. (1998) Gen. Pharmacol. 30:777-782; in document “Arthogen-CIA Reagents and Consulting for Arthritis Research” (2002) Chondrex Inc.). Other mammals can be used for induction of arthritis including but not limited to, rats and monkeys (Yoo et al. (1988) J. Exp. Med. 168:777-782; Larson et al. (1990) Arthritis Rheum. 33:693-701; Griffiths et al. (1988) Intern. Rev. Immunol. 4:1-15; Griffiths et al. (1992) J. Immunol. 149:309-316: in document “Arthogen-CIA Reagents and Consulting for Arthritis Research” (2002) Chondrex Inc.).

In the present invention treatment of mice with HN compounds during the development of CIA reduces the severity of paw inflammation as compared to untreated mice. Inflammation can be detected using any method known to those of skill in the art. For example, qualitative scoring of swelling and redness has been used to assess the degree of arthritis (referred to as arthritis score). This is determined by macroscopic evaluation of symptoms (Wallace et al. (1999) J. Immunol. 162:5547-5555; Kim et al. (2002) Arthritis Rheum. 46:793-801; e.g. example below) or by measuring paw thickness using a caliper or a plethysmometer (Wallace et al. (1999) J. Immunol. 162:5547-5555; Takaoda et al. (1998) Gen. Pharmac. 30:777-782; as in example below). Inflammation can be detected by computed tomography (CT), magnetic resonance imaging (MRI), ultrasonography or scintigraphic imaging.

In other embodiments of the present invention, treatment of CIA-induced mice with HN compounds reduces the histopathological changes associated with inflammatory and destructive arthritis as compared to untreated mice. Histological changes of arthritis can be assessed using methods described herein in the example below and in the literature. For example, histological assessment of arthritis in joints of HN compounds-treated and non-treated mice, can be done on stained tissue sections of joints using defined pathological features graded for severity based on a scoring system in a blinded manner. These features may include soft tissue infiltrate, synovitis, joint exudates, pannus, cartilage destruction and bone erosion (Staite et al. (1990) Arthritis Rheum. 33:253-260; Lawlor et al. (2001) Clin. Exp. Immunol. 119:361-367; Wallace et al. (1999) J. Immunol. 162:5547-5555; Kim et al. (2002) Arthritis Rheum. 46:793-801; Campbell et al. (2001) J. Clin. Invest. 107:1519-1527; and as described in example below). Based on the aforementioned histologic criteria, joints are classified as demonstrating inflammatory arthritis as defined by increased score of tissue infiltrate, synovitis and joint exudates, or destructive arthritis as defined by higher scores for pannus formation cartilage degradation or bone degradation (Lawlor et al. (2001) Arthritis Rheum. 44:442-450; and as described herein in example below).

In another embodiment, treatment in vivo with HN compounds reduces both P2X₇-induced IL-1.beta release in peripheral blood, and IL-1.beta levels in joints of CIA-induced mice as compared to untreated animals. Increased production of IL-1.beta has been demonstrated in the circulation of patients with RA (Chikanza et al. (1995) Arthritis Rheum. 38:642-648) and the levels of measured IL-1.beta have been shown to correlate with disease severity in RA (Eastgate et al. (1988) Lancet 2:706-708; Rooney et al. (1990) Rheumatol. Int. 10:217-219). Elevated levels of IL-1.beta have been detected in the synovium, synovial fluid, cartilage and bronchalveolar lavage of patients with RA (Firestein et al. (1992) Arthritis Rheum. 149:1054-1062; Kahle et al. (1992) Ann. Rheum. Dis. 51:731-734; Koch et al. (1992) Clin. Immunol. Immunopathol. 65:23-29). As mentioned above, ATP acting at the P2X₇ receptor is required for the generation of high levels of extracellular IL-1.beta and mice deficient in P2X₇ receptor showed a reduced severity of arthritis in an anti-collagen antibody arthritis model (Labasi et al. (2002) J. Immunol. 168:6436-6445). Examples of methods for the measurement of P2X₇-induced IL-1.beta release in peripheral blood and for determination of IL-1.beta levels in joints can be found in the art. For example, whole blood from HN-treated compounds and non-treated mice is collected in heparin containing tubes and kept on ice. Blood diluted in culture medium (1:1) is incubated first with LPS and subsequently with ATP as a secretion stimulus (Perregaux et al. (2000) J. Immunol. 165:4615-4623 and as in example herein). Whole mouse knees or ankles from HN-treated and non-treated mice are snap frozen in liquid nitrogen, ground into powder and tissue lysates are prepared for measurement of cytokine (Kim et al. (2002) Arthritis Rheum. 46:793-801; as further described in example below). Levels of IL-1.beta can be measured by ELISA assays.

The present invention includes compositions that may conveniently be presented in unit dosage form and may be prepared by any methods well known in the art of pharmacy. The HN compounds of the present invention can be combined with a pharmaceutically acceptable adjuvant, carrier, diluent, or excipient to produce a single dosage form, which amount may vary depending upon factors such as the host being treated and the particular mode of administration. Generally the amount of the compound of the invention may be in the range from about 1% to about 99% of the composition, preferably about 5% to about 70%, most preferably from about 10% to about 30% although the amount of the dose and the mode of administration will vary depending upon numerous factors such as the host being treated in a particular mode of administration.

An appropriate dose may be from about 5 mg to about 50 mg per person per day. The exact dose will be determined empirically.

Suitable methods of administration of the compounds of the present invention, and the compositions referred to above, include oral, nasal, topical (including buccal and sublingual), rectal, vaginal and/or parenteral administration, including injectable.

The compound of the present invention can be administered topically in the form of solutions, suspensions, aerosols and dry powdered formulations; systemically in the form of tablets, capsules, syrups, powders or granules; by parenteral administration in the form of solutions or suspensions; by subcultaneous administration; by rectal administration in the form of suppositories; or transdermally.

The compounds of the present invention may be formulated for parenteral administration by injection, including using conventional catheterization techniques or infusion. Formulations for injection may be presented in unit dosage form, e.g., in ampoules or in multi-dose containers, with added preservatives. The compositions may take such forms as suspensions, solutions or emulsions in oily or aqueous vehicles. The formulations may also contain formulating agents such as suspending, stabilizing and/or dispersing agents. The compounds of the present invention may also be in a dry form for reconstitution with a suitable vehicle prior to use. Various terms used herein are defined as stated below.

By “reduction” is meant a statistically significant decrease (p<0.05).

By “P2X₇ receptor-stimulated pore formation” is meant an increase in ATP or BzATP-stimulated cell permeability and an increased accumulation of a macromolecule, e.g. a dye macromolecule such as ethidium bromide.

By “P2X₇ receptor-stimulated IL-1. alpha. IL-1. beta., and IL-18 production and IL-6 release in animal cells” is meant an increase in ATP-stimulated IL-1.alpha, IL-1.beta, IL-18 production and IL-6 release in activated inflammatory cells (e.g., activated by lipopolysaccharide treatment or cytokine pretreatment, such as tumor necrosis factor-alpha).

By “P2X₇-stimulated IL-1.beta. IL-alpha and IL-18 production and IL-6 release in mammals” is meant an increase in ATP-stimulated IL-1.beta production and IL-6 release in inflammation-induced cells.

The term “P2X₇-stimulated IL-1.beta, IL-1.alpha and IL-18 production” is meant to include posttranslational processing and release.

The term “animal cell” is meant to include a cell in an immortalized cell line, in a cell line transfected with a gene, in a primary cell preparation, or otherwise derived from an animal. Preferably, the animal is a mammal such as a human, mouse, or rat.

The present invention will be further illustrated in the following examples.

### EXAMPLES

The following examples are provided for illustrative purposes only and are not to be construed as limiting the present invention in any manner.

### Materials and Methods

Alveolar Macrophage Isolation

Male Wistar rats weighing 225-250 g were purchased from Charles River Canada, Inc. (St-Constant QC). These animals were shipped behind filter barriers and housed in isolated temperature-controlled quarters in an animal isolator unit (Johns Scientific Inc., Toronto, Ontario). They were given standard lab chow and water ad libitum, and were used within two weeks.

Alveolar macrophages (AM) were recovered from normal rats by bronchoalveolar lavage (BAL) as described previously (Lemaire (1991) Am. J. Pathol. 138:487-495). The lungs were lavaged with 7 ml-aliquots of sterile phosphate buffered saline (pH 7.4; Wisent, St. Bruno, QC), and BAL cells were obtained by centrifugation at 200×g at 4° C. for 5 minutes. The cells were resuspended in RPMI 1640 medium (Wisent, St-Bruno, QC) supplemented with 0.5% dialysed fetal bovine serum (Wisent. St-Bruno, QC), 0.005% gentamycin (Schering Canada Inc. Pointe Claire. QC) and 0.8% HEPES (Sigma Chemical Co., St-Louis, MO) which will henceforth be referred to as complete culture medium (CM). Cells were counted in a hemocytometer chamber, and cell viability (>98%) was determined by trypan blue exclusion. Differential analysis of lavage cells made from cytocentrifuge smears (Shandon, 2.5×10⁴ cells) stained with Wright-Giemsa indicated that 99% of the BAL cell population was of macrophage morphology.

ATP-induced membrane permeabilization and pore formation

Freshly isolated AM (1×10⁵) were incubated in 200 μl complete medium in 96 wells tissue culture plates for 24 h. Following centrifugation, medium was replaced with saline solution containing 125 mM NaCl, 5 mM KCL, 1 mM MgSO₄, 1 mM Na₂HPO₄, 5.5 mM glucose, 5 mM NaHCO₃, 1 mM CaCl₂ and 20 mM Hepes, pH 7.6. Ethidium bromide was added to a final concentration of 20 μM and AM were stimulated with ATP (5 mM, Sigma), 3′-O-(4-benzoylbenzoyl ATP (BzATP) (0.5 mM, Molecular Probes) or uridine 5′-triphosphate (UTP) (Sigma), at 37° C. for 5 minutes. In some experiments, cells were pre-treated with oxidized ATP (oATP, 0.1 mM, Sigma) for 2 h or Brilliant Blue G (BBG, 5×10⁻⁹M, Sigma) for 5 minutes, or HN compounds for 5 minutes before ATP stimulation. After ATP stimulation, the plate was centrifuged (200×g, 1 minute), culture supernatants were replaced with fresh buffer and the fluorescence was analyzed using a Zeiss Axiovert S1100 TV inverted microscope equipped with a rhodamine filter and a 32× objective. Images were captured and analysed with the Northern Eclipse image analysis software. Data are expressed as percentage of cells that become permeabilized to ethidium bromide.

ATP-induced Apoptosis and Cell death Assays

Macrophage apoptosis was examined by a combination of morphological differential staining and the detection of DNA fragmentation in the cells. For morphological differential staining. 1×10⁵ AM were incubated in Lab Tek tissue culture chambers (Nunc. Naperville, Ill.) in 200 μl of complete medium alone or with ATP (5 mM) in the presence and absence of HN compounds for 3 h at 37° C. in 5% CO₂. The medium was then replaced with PBS containing 1.5% BSA and cells were incubated with Hoechst-33342 (0.2 μg/ml) (Molecular Probes) for 10 min followed by propidium iodide (0.02 μg/ml) for 10 min on ice, protected from light. Cells were then examined for apoptosis (increased nuclei fluorescence using a Hoechst filter (Zeiss)) without secondary necrosis (i.e. without nuclei labeled with propidium iodide) and the percentage of apoptotic AM was determined.

Detection of histone-associated DNA fragments which demonstrates the internucleosomal degradation of genomic DNA occurring during apoptosis was performed using a cell death ELISA kit (Boehinger Mannhein) according to the manufacturer's protocol. This assay using mouse monoclonal antibodies directed against DNA and histones respectively, allows the quantitative determination of mono- and oligonucleosomes in the cytoplasmic fraction of cell lysates prepared from macrophages (Li et al. (1997) Toxicol. Appl. Pharmacol. 145:331-339). Alveolar macrophages (1×10⁵) were incubated in 200 μl complete medium in 96 wells tissue culture plates with ATP (5 mM) in the presence and absence of HN compounds for 30 minutes and media was replaced with fresh culture media. After 24 h, the plate was centrifuged, the culture supernatants removed and macrophages were lysed in lysis buffer for 30 min on ice and centrifuged at 14,000 rpm for 15 min at 4° C. Cytoplasmic oligonucleosomal DNA was measured in cell supernatants by ELISA.

Cell death was assessed by the release of the cytoplasmic enzyme lactate dehydrogenase (LDH), a reliable measure of P2X₇-induced cell death (Ferrari et al. (1999) FEBS Lett. 447:71-75). Alveolar macrophages (0.5×10⁶) were incubated in 1 ml complete medium in 24 wells tissue culture plates for 24 h. The medium was then replaced with 250 μl fresh complete medium and cells were incubated initially with lipopolysaccharide (LPS, 1 μg/ml) (Sigma) for 2 hours followed by a 1 hour incubation with ATP (5 mM. Sigma) or 30 minutes incubation with Nigericin (20 μM) in the presence and absence of HN compounds (10⁻⁹M). The plate was then centrifuged at 200 xg for 5 minutes, and 100 μl of cell-free supernatants were collected and tested for LDH using a cytotoxicity detection kit for LDH (Roche Diagnostic, Laval Qué.).

ATP-induced extracellular release of IL-1. beta, and IL-1. alpha from macrophage in in vitro.

Alveolar macrophages (1×10⁶ cells/ml) were incubated in complete medium in 96 wells tissue culture plates overnight at 37° C. in 5% CO₂. The medium was changed for fresh medium and cells were incubated with Lipopolysaccharide (LPS. 1 μg/ml) (Sigma) for 3 h followed by ATP (5 mM) or 3′-0-(4-benzoylbenzoyl ATP (BZATP) (0.5 mM. Molecular Robes) for 1 h in the presence and absence of HN compounds (10⁻⁹M and 10⁻⁸M) or Brilliant Blue G (BBG, 5×10⁻⁹ M, Sigma). In some experiments, cells were pre-treated with oxidized ATP (oATP, 100 μM, Sigma) for 2 h before LPS and ATP or BzATP stimulation. In other experiments, cells were stimulated with LPS (1 μg/ml) for 3 h and subsequently with Nigericin (20 μM) for 30 min in the presence and absence of HN compounds (10⁻⁹, 10⁻⁸M). Controls were incubated with LPS only. After incubation, cell culture media were collected, centrifuged in a microfuge (Eppendorf) and the cell-free supernatants were collected and stored at −80° C. for cytokine measurement. Levels of immunoreactive IL-1.beta and IL-1.alpha were assayed using ELISA (R&D Systems, Minneapolis, Minn. and Biosource (Medicorp), Camarillo, Calif. respectively) performed according to the manufacturer's protocol.

### Phosphatidylserine Translocation (PS Flip Assay)

Alveolar macrophages (10⁵) were plated in Lab Tek tissue culture chambers and incubated overnight in complete medium at 37° C. in 5% CO₂. After 24 h, the culture medium was replaced with 200 μl of incubation buffer (125 mM NaCl, 5 mM Kcl, 1 mM mg SO₄. 1 mM Na_{z}H PO₄, 5.5 mM NaHCO₃, 1 mM CaCl₂ and 20 mM Hepes, pH 7.6) and cells were incubated in the presence and absence of HN compounds for 5 minutes. Fluorescein-conjugated annexin-V (Molecular Probes) was then applied to cells prior to P2X₇ receptor activation with 3′-0-(4-benzoylbenzoyl ATP (BZATP) (100 μM) for 5 minutes. As control cells were labeled with fluorescence-conjugated annexin-V for 5 minutes and incubated in the absence of BzATP for an additional 5 minutes. Following rapid centrifugation (1 min. 200 xg) culture supernatants were replaced with 200 μl of fresh incubation buffer pH 7.6 and fluorescent annexin-positive cells were analyzed using a Zeiss Axiovert S1100 TV inverted microscope equipped with a FITC filter and 32× objective. Images were captured and analysed with the Northern Eclipse image analysis software.

ATP-Induced Extracellular Release of IL-1.Beta. IL-1.Alpha and IL-6 In Vivo

Groups of mice were injected intraperitoneally (i.p.) with 50 μg of LPS. Two hours after this LPS injection, mice were injected i.p. with either PBS or ATP (5 mM, adjusted to pH 7), or with HN compounds (0.1 mg and 0.3 mg) 15 minutes before ATP injection. Mice were sacrificed 30 minutes (IL-1.beta and IL-1.alpha) or 120 minutes (IL-6) after the ATP or PBS injection and peritoneal cavities were lavaged each with 3 ml PBS. Samples of peritoneal lavages were centrifuged, cell-free supernatants were collected and tested for the presence of IL-1.beta, IL-1.alpha and IL-6 using ELISA (R&D Systems, Minneapolis, Minn., and Biosource, Camarillo, Calif., respectively).

Collagen-Induced Arthritis (CIA)

For induction of arthritis, DBA/1 mice (The Jackson Laboratory, Bar Harbor, Me.) were immunized intradermally at the base of the tail with bovine type II collagen (100 μg; Chondrex, Richmond, Wash.) emulsified in Freund's complete adjuvant (Chondrex, Richmond, Wash.). On day 21, the animals were boosted with an intradermal injection of 100 μg type II collagen. To synchronize the onset of arthritis, mice were subsequently injected i.p. with LPS (50 μg) (Yoshino et al. (2000) J. Immunol. 163:3417-3422). With this procedure, arthritis will develop 3 to 7 days before the desired onset of arthritis. Following this immunization protocol, 80% of the mice develop arthritis which was monitored for 4 weeks as described below.

To test the subject of the present invention, mice were divided in various groups: mice injected with saline (negative control), mice injected with collagen and LPS (positive control), mice injected with collagen and treated i.p. daily with HN compound starting 2 days before i.p. injection of LPS and onset of arthritis, and mice injected with collagen and LPS, and treated i.p. daily with RN compound after the onset of arthritis as monitored by the presence of a positive clinical score index as described below.

The incidence of arthritis was monitored daily by macroscopic clinical scoring on a 0-3 scale where 0=normal: 1=slight swelling and erythema. 2=pronounced swelling; 2.5=maximum swelling and edema of entire paw including digits; 3=joint rigidity. Each limb was graded, resulting in a maximal clinical score of 12 per animal (Malfait et al. (2000) Proc. Natl. Sci. USA 97:9561-9566, Campbell et al. (2001) J. Clin. Invest. 107:1519-1527). Limb swelling was also measured with calipers (Wallace et al. (1999) J. Immunol. 162:5547-5555). For histologic assessment of arthritis, the mice were killed, the knee joint was excised, fixed in formalin and decalcified in a solution containing 15% Formic acid+2% acetic acid, 5% Ammonia hydroxide and 5% amonia oxalit (1:1:1). The paws were embedded in paraffin, sectioned (5 μL thick) and stained with hematoxylin and eosin. The assessment of arthritis was performed on coded knee sections by an observer blinded to the experimental groups. Knee sections were graded 0 (normal) to 3 (severe) for the severity of 6 components of arthritis based on the scoring system of Staite et al. (Arthritis Rheum. (1990) 33:253-260). Soft tissue inflammation was evaluated in the infrapatellar fat pads, joint capsule and the area adjacent to periosteal sheath and graded according to the extent of cellular infiltration. Synovitis was defined as hyperplasia of the synovial lining layer. Pannus was defined as hypertrophic synovial tissue forming a tight junction with the articular surface. Joint space exudate was identified as neutrophils and macrophages interspersed with fibrin-like material in the joint space. Cartilage destruction was evaluated as the extent of loss of glycosaminoglycan matrix, death of chondrocytes, thinning and destruction. Bone degradation was evaluated as the extent and depth of subchondral and subperiosteal bone erosion. Based on the above histologic scores, joints were classified as demonstrating inflammatory arthritis if there was a joint space exudate score of 1 or more or a soft tissue infiltrate score of 1 or more combined with a synovitis score of 2 or more. Destructive arthritis was defined for joints that scored 2 or more for pannus or 1 or more for either cartilage degradation or bone degradation (Lawlor et al. (2001) Arthritis Rheum. 44:442-450).

IL-1.Beta Analysis in CIA

Paws from animals in each group were snap frozen in liquid nitrogen and were grounded into powder with a pre-cooled mortar pestle, then lysed with lysis buffer (25 mM Tris HCl, 50 mM NaCl, 0.5% sodium deoxycholate. 2% Nonidet P40, 0.2% sodium dodecyl sulfate. 1 mM phenylmethylsulfonyl fluoride). The samples were subjected to centrifugation (13,000 rpm for 10 minutes) and the resulting supernatants were analysed for protein content (Bio Rad) and IL-1.beta was measured by ELISA (R&D Systems. Minneapolis, Minn.).

P2X₇ receptor-stimulated IL-1 release ex vivo was performed with a blood-based IL-1.beta assay (Perregaux et al. (2000) J. Immunol. 165:4615-4623). Blood was collected from mice in each group in heparin-containing tubes. A total of 120 μl of blood was placed into an individual well of a 96-well plate and diluted with 120 μl RPMI 1640 medium containing 25 mM HEPES and 1% dialysed FBS. The diluted blood samples were incubated for 3 h in the presence and absence of LPS (1 μg/ml) at 37° C. in 5% CO₂. ATP (5 mM, pH 7) was then added as a secretion stimulus and the mixtures were incubated at 37° C. for an additional 2 h. The 96-well plates were then centrifuged at 700×g for 10 min at 4° C., and the resulting plasma samples were collected and assayed for their IL-1.beta content by ELISA (R&D Systems, Minneapolis, Minn.).

### Results

Membrane Permeabilization and Pore Formation

A hallmark of the P2X₇ receptor is its ability to facilitate cellular uptake of organic molecules ≦900 daltons such as the fluorescent dye ethidium bromide in response to ATP activation (Falzoni et al. (1995) J. Clin. Invest. 95:1207-1216; Alcaraz et al. (2003) Biorg. Med. Chem. Lett. 13:4043-4046).

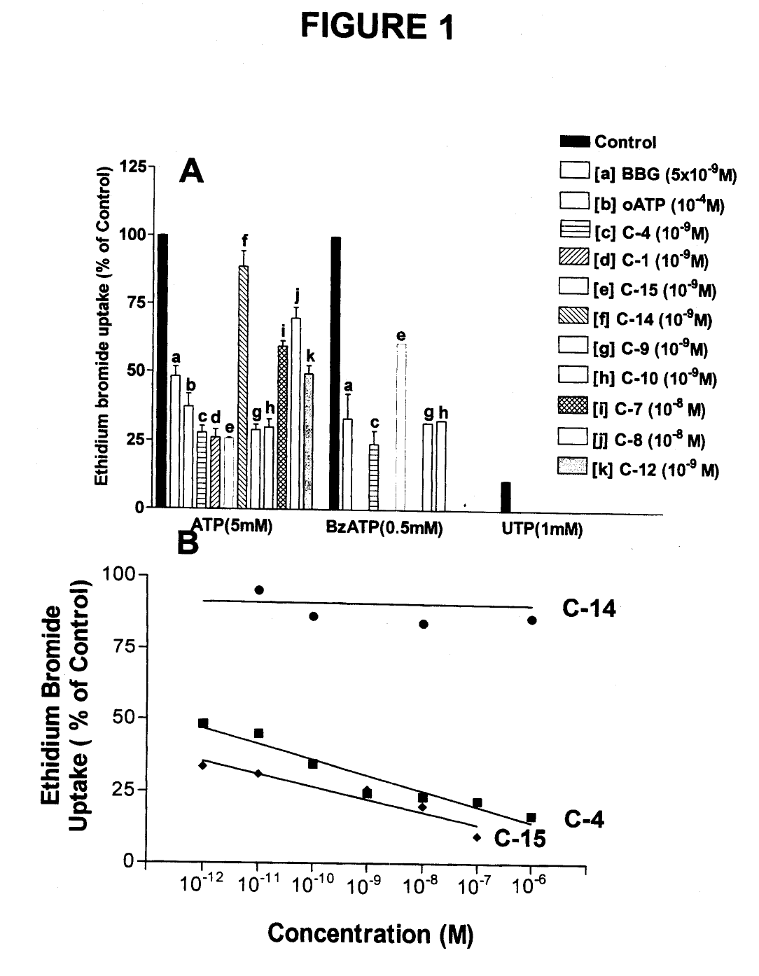

Rat alveolar macrophages treated for 5 minutes with ATP (5 mM) or BzATP (0.5 mM), a more potent agonist at the P2X₇ receptor, became permeabilized to ethidium bromide which could be seen throughout the cytoplasm of treated macrophages upon microscopic observation. Under these conditions, approximately 50% of macrophages became permeabilized to ethidium bromine while dye uptake was not significantly observed in control cells or cells incubated with UTP, a nucleotide known to be inactive at the P2X₇ receptor (Charlton et al. (1996) Br. J. Pharmacol. 19:1301-1303). Pre-treatment of macrophages with the P2X₇ receptor antagonists, oATP (Murgia et al. (1993) Biochem. J. 288:897-901) and Brilliant Blue G (Jiang et al. (2000) Mol. Pharmacol. 58:82-88) blocked ATP- and BzATP-mediated peimeabilization. Similarly, the HN linear peptides C-4 and C-1, the cyclic peptides C-7, C-8, C-9 and C-10 and the non-peptide C-12 and C-15 (10⁻⁹M) all inhibited significantly ATP-induced pore formation. In contrast. HN-like non peptide C-14. the 2S isomeric form of C-15, and a negative control, was inactive (FIG. 1A and FIG. 1B). The effects of HN compounds C-4, C-9, C-10 and C-15 were also tested on the response to BzATP and found to inhibit as well BzATP-induced pore formation.

As shown in FIG. 1B, HN compounds C-4 and C-15 inhibited ATP-induced pore formation over a wide range of concentrations (10⁻¹² M to 10⁻⁶ M) in a dose-dependent manner whereas C-14 was inactive at all doses tested.

Apoptosis and Cell Death

ATP-induced cell apoptosis is a striking effect of P2X₇ receptor activation and has been used as a measure of P2X₇ receptor activity (Ferrari et al. (1997) Neuropharmacol. 36:1295-1301).

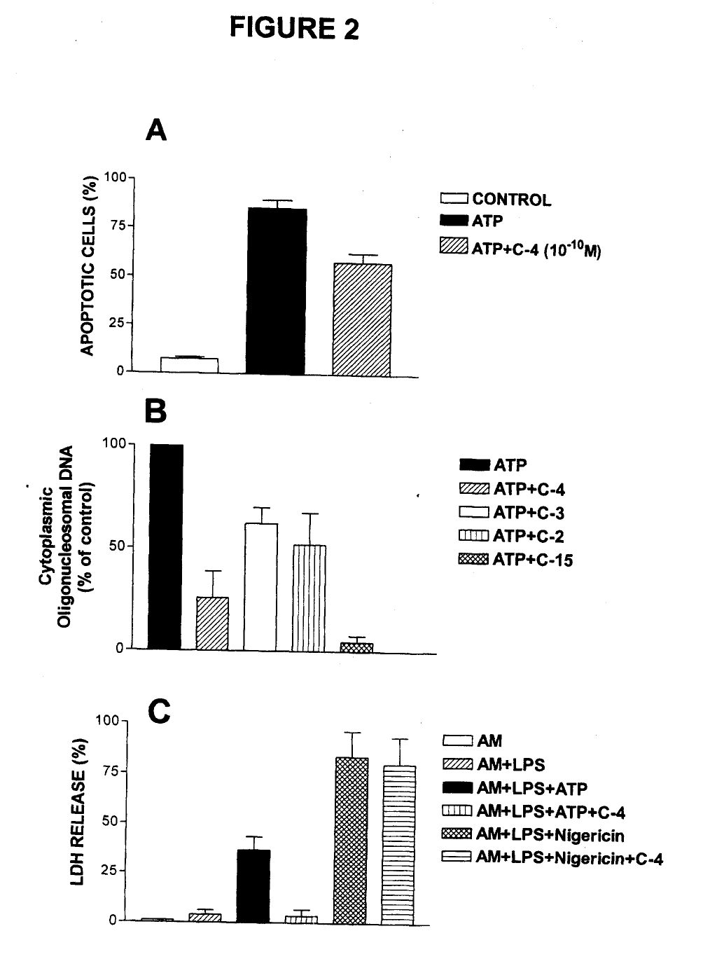

As shown in FIG. 2A, ATP induced apoptosis in approximately 85% of macrophages compared to 7% in controls and 54% in cells treated with the linear HN peptide C-4 (10⁻¹⁰M) as determined by morphological differential staining with Hoechst and propidium iodide. Inhibition of ATP-induced apoptosis by HN compound C-4 was confirmed by cell death ELISA assay shown to be selective for apoptotic DNA fragmentation in alveolar macrophages (Li et al. (1996) Toxicol. Appl. Pharmacol. 145:331-339). A significant decrease in cytoplasmic oligonucleosomal DNA was seen in macrophages incubated with C-4 (10⁻⁹M) and ATP in contrast to macrophages incubated with ATP alone (FIG. 23B). In addition, other HN compounds (10⁻⁹M) including linear peptides C-2 and C-3 and the non-peptide C-15 also inhibited cytoplasmic oligonucleosomal DNA with the following rank order: C-15>C-4>C-2>C-3.

Activation of the P2X₇ receptor by ATP in macrophages primed with LPS induces the release of the cytoplasmic enzyme LDH, an indicator of cell death (Ferrari et al. (1999) FEBS Lett. 447:71-75). FIG. 2C further illustrates that UN linear peptide C-4 inhibited significantly ATP-induced release of LDH from macrophages stimulated with LPS. Such response is strictly dependent on P2X₇ receptor activation since LPS-stimulated macrophages from P2X₇ knockout mice are resistant to ATP-induced cell death and failed to release LDH in response to ATP (Brough et al. (2002) Mol. Cell Neurosci. 19:272-280). By contrast, HN linear peptide C-4 did not inhibit LDH release in response to the potassium inonophore Nigericin which acts independently of the P2X₇ receptor (Solle et al. (2001) J. Biol. Chem. 276:125-132) (FIG. 2C). Therefore, UN compounds inhibit selectively cell death induced by P2X₇ receptor activation.

ATP-Induced Release of IL-1.Beta and IL-1.Alpha in Macrophages

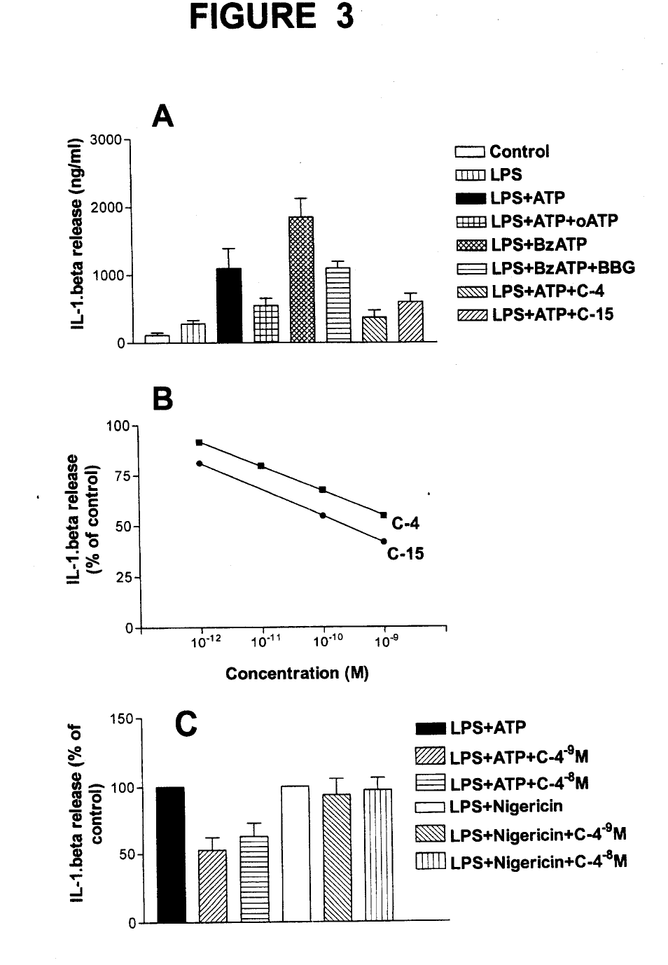

Extracellular release of significant levels of IL-1.beta by alveolar macrophages requires that LPS-primed macrophages receive ATP or BzATP as a secretion stimulus (FIG. 3A), and such response requires the activation of P2X₇ receptor (Solle et al. (2001) J. Biol. Chem. 276:125-132). As expected, ATP- and BzATP-induced IL-1.beta release by LPS-primed alveolar macrophages were significantly reduced by pre-treatment with the P2X₇ receptor antagonists oATP and Brilliant Blue G (BBG), respectively (FIG. 3A). In parallel cultures of LPS-primed macrophages incubated with UN linear peptide C-4 or HN non-peptide C-15 prior to ATP or BzATP stimulation, a significant inhibition of extracellular IL-1.beta release was achieved at a concentration of (10⁻⁹ M) (FIG. 3A). Inhibition was already seen at 10⁻¹²M and was dose-dependent with a half-maximal response obtained approximately at 10⁻⁹M for C-4 and 10⁻¹⁰ M for C-15 (FIG. 3B). By contrast, 1N linear peptide C-4 at concentration of 10⁻⁹M and 10⁻⁸M, had no effect on IL-1.beta release induced by Nigericin, a P2X₇-independent stimulus (FIG. 3C). Therefore, HN compounds inhibit extracellular release of mature and bioactive IL-1.beta triggered through P2X₇ receptor activation.

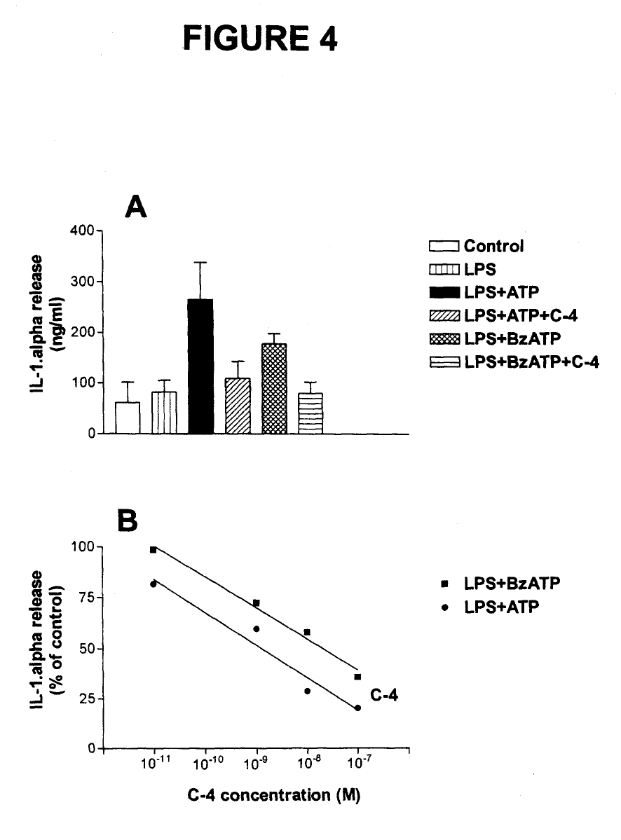

ATP and BzATP also act as secretion stimuli for the release of Il-1.alpha. When LPS-primed macrophages were incubated for 15 minutes with HN linear peptide C-4 prior to ATP or BzATP stimulation, a dose-dependent inhibition of extracellular release of IL-1.alpha was observed (FIG. 4A; FIG. 4B).

Phosphatidylserine Translocation (PS Flip)

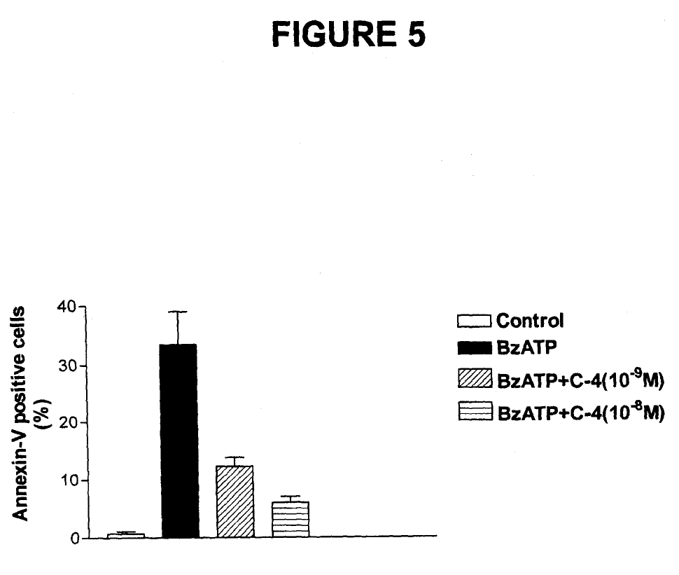

Brief (≦10 minutes) activation of the P2X₇ receptor induces phosphalidylserine translocation from the inner leaflet of the plasma membrane to the outer. Under these conditions PS flip is reversible and has been associated with rapid release of the cytokine (MacKenzie et al. (2001) Immunity 8:825-835). As illustrated in FIG. 5, pre-incubation of macrophages with HN compound C-4 for 5 minutes at 10⁻⁹ M or 10⁻⁸M, inhibited PS flip in response to BzATP as determined by a significant decrease in the number of annexin-V positive cells (from 32% for untreated BzATP-stimulated macropages to 12% and 5% for macrophages treated respectively with 10⁻⁹ M and 10⁻⁸M HN linear peptide C-4.

ATP-Induced Extracellular Release of IL-1.beta, IL-1.alpha and IL-6 In Vivo

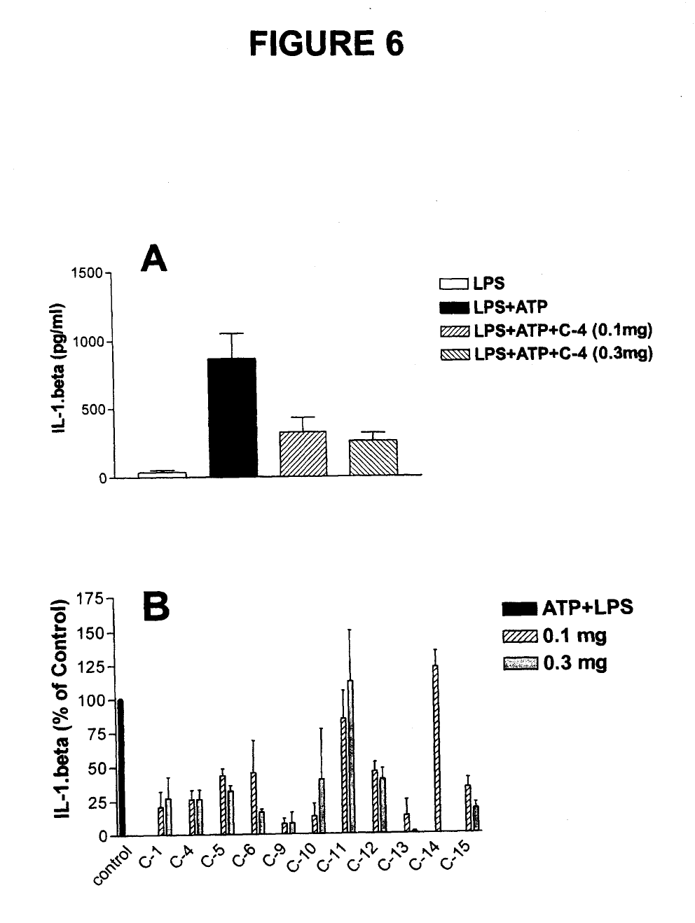

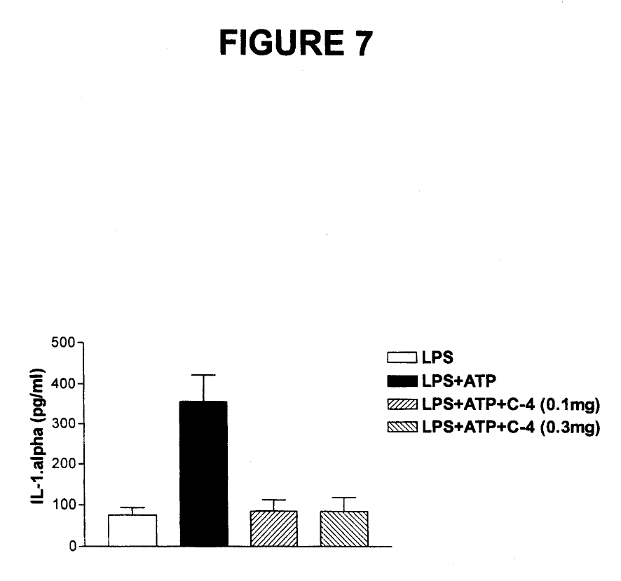

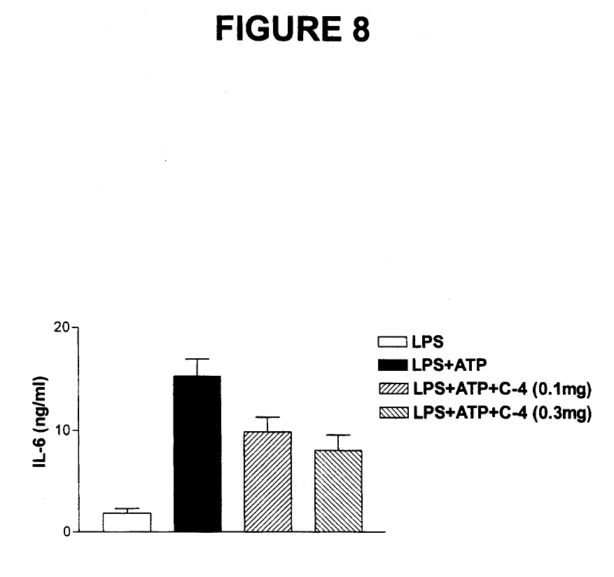

Macrophages exposed to LPS in vivo also require a secretion stimulus such as ATP to elicit efficient externalization of mature IL-1.beta (Griffiths et al. (1995) J. Immunol. 154:2021-2028). Such requirement is strictly dependent upon activation of the P2X₇ receptor since P2X₇−/− knock-out mice primed i.p. with LPS and subsequently challenged with ATP, failed to release detectable levels of IL-1.beta and IL-1 alpha and released reduced levels of extracellular IL-6 (Solle et al. (2001) J. Biol. Chem. 276:125-132). Mice were primed with LPS and 2 hours later they received an i.p. injection of PBS or ATP. To determine whether HN compounds affect ATP response, mice were treated i.p. with HN compounds (0.1 mg and 0.3 mg) 15 minutes prior to i.p. injection of ATP. Peritoneal lavage fluids from these mice then were assessed for IL-1.beta content by ELISA. While LPS-primed mice yielded no significant IL-1.beta in response to PBS, LPS-primed mice generated significant levels of IL-1.beta in response to ATP challenge (FIG. 6A). In contrast. LPS-primed mice treated i.p. with 0.1 mg or 0.3 mg HN linear peptide C-4 generated significantly reduced levels of IL-1.beta in response to ATP challenge (FIG. 6A). Similarly, LPS-primed mice treated i.p. with either the linear peptides C-1, C-4, C-5 or C-6. the cyclic HN peptides C-9 or C-10 or the non-peptides C-12 C-13 or C-15, produced reduced levels of IL-1.beta in response to ATP (FIG. 6B). In contrast, HN-like non peptide C-11, the 2S isomeric form of C-12 and a negative control, and C-14, the 2S isomeric form of C-15 and a negative control were inactive (FIG. 6B). Treatment with HN linear peptide C-4 also reduced significantly the levels of IL-1.alpha in peritoneal lavages of mice primed with LPS and stimulated with ATP (FIG. 7). As noted earlier. IL-1 signaling often leads to the production of other cytokines such as IL-6 (Allen et al. (2000) J. Exp. Med. 191:859-869). To demonstrate whether HN compounds inhibit this cascade effect, mice were treated i.p. with HN linear peptide C-4 (0.1 mg and 0.3 mg) 15 minutes prior to i.p. injection of ATP. Controls include mice primed with LPS for 2 h and challenged with an i.p. injection of ATP (or PBS) to promote IL-1 posttranslational processing. Peritoneal lavages were collected 2 h after ATP injection and analyzed for IL-6 by ELISA. Mice that received an initial priming injection of LPS followed by PBS yielded low levels of IL-6 at 2 h (2-3 ng/ml). On the other hand, LPS-primed mice that subsequently were challenged with ATP demonstrated a dramatic increase in lavage fluid IL-6 (16 ng/ml) (FIG. 8). In contrast, samples recovered from mice treated with 0.1 mg and 0.3 mg HN linear peptide C-4 contained reduced levels of IL-6 (9.5 and 7 ng/ml respectively) (FIG. 8). Overall, it could be appreciated from the results illustrated in FIG. 6A, FIG. 6B and FIG. 8, that LPS-primed mice that received a subsequent challenge with ATP displayed increased levels of IL-1.beta in peritoneal lavage fluids 30 minutes later followed at 2 h by increases of IL-6 levels demonstrating that a IL-1-IL-6 cascade effect occurred in vivo. Moreover, results illustrated in these Figures also demonstrate that i.p. treatment with HN compounds inhibit significantly this cascade effect.

Collagen-Induced Arthritis (CIA) in Mice

The effects of HN compounds treatment was examined in a murine model of arthritis in which P2X₇ receptor (Labassi et al. (2002) J. Immunol. 168:6436-6445) and pro-inflammatory IL-1 (reviewed in Braddock et al. (2004) Nature Rev. 3:1-10) have been previously shown to play key roles. The mouse CIA model used to test HN compounds develops histopathological features of inflammatory arthritis that resemble those of human rheumatoid arthritis (RA) (Luros et al. (2001) Immunology 103:407-416) and has been the model of choice to test potential new therapeutic agents for the treatment of human RA. To induce inflammatory arthritis mice were administered collagen followed by LPS to enhance and synchronize classic CIA (Yoshimo et al. (2000) J. Immunol. 163:3417-3422; in Chondrex Inc. document. “Arthogen CIA reagents and consulting for arthritis research” (2002)). This protocol induces arthritis 3 to 7 days following LPS injection. In a first set of experiments. HN compound treatment was initiated at day 44 following primary immunization and 2 days before LPS injection, and continued daily up to day 61.

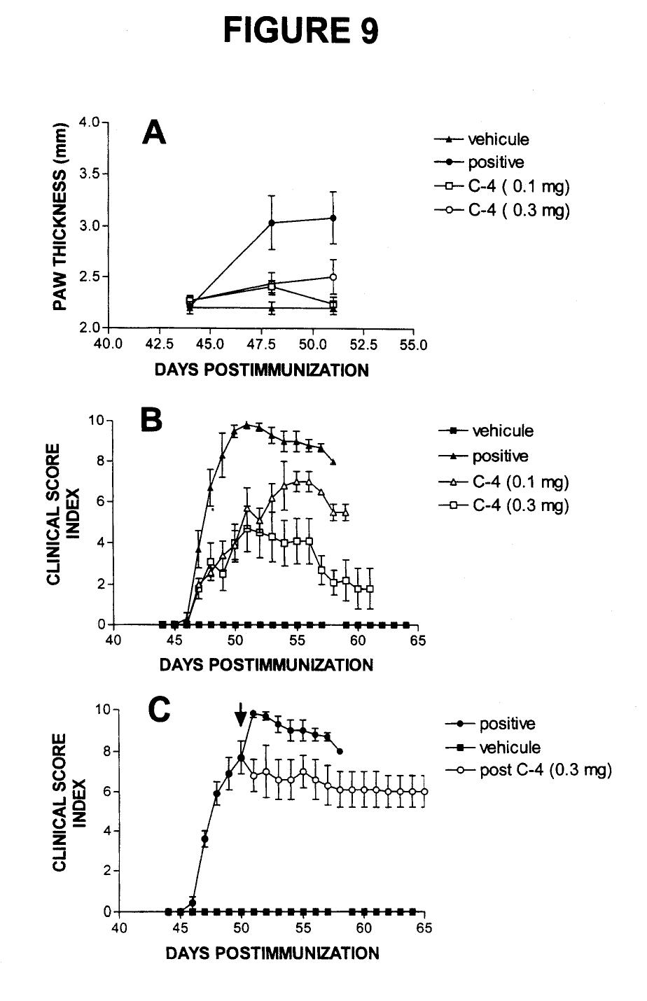

Treatment with HN linear peptide C-4 (0.1 and 0.3 mg, i.p.) inhibited the severity of arthritis as shown by a reduction of limb swelling (FIG. 9A) and of the arthritis score index (FIG. 9B) compared to untreated mice. A maximal arthritis index of 4.5 was observed in mice treated with C-4 (0.3 mg) compared to 9.5 for untreated animals. In a second set of experiments, HN compound C-4 was administered after joint inflammation was clearly established. At day 50 post-immunization, mice with an arthritis score index of 7.7 were injected daily with HN linear peptide C-4 (0.3 mg, i.p.) up to day 65 post-immunization. The mean arthritis index over that 15 day-period was 6.2±0.07 in mice treated with C-4 while it increased to 9.1±0.18* (p<0.01) in untreated animals (FIG. 9C).

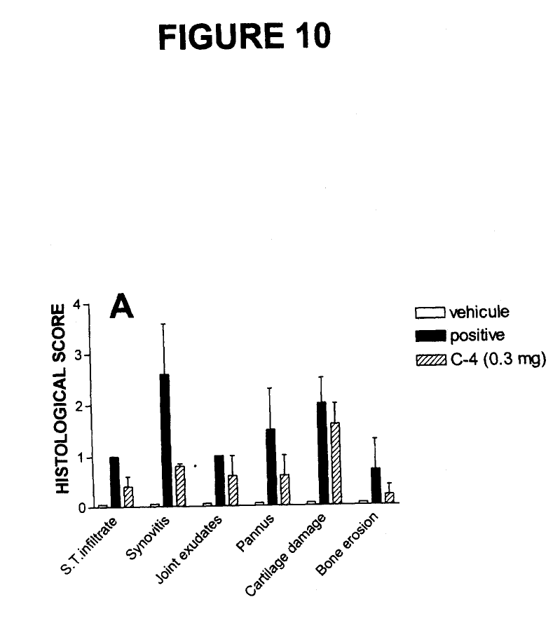

At sacrifice, histologic assessment of arthritis was performed on knee sections graded 0 (normal) to 3 (severe) for six components of arthritis: soft tissue infiltrate/inflammation, synovitis, joint exudates, pannus, cartilage damage and bone destruction. Based on these histologic scores, joints were classified as demonstrating inflammatory arthritis if there was a joint space exudate score of ≧1 or a soft tissue infiltrate score ≧1 combined with a synovitis score ≧2. Destructive arthritis was defined for joints that scored ≧2 for pannus or >1 for either cartilage degradation or bone erosion (Lawlor et al. (2001) Arthritis Rheum. 44:442-450). Histologic examination showed that treatment with HN linear peptide C-4 (0.3 mg, i.p.) reduced inflammatory arthritis as evidenced by lower score for soft tissue infiltrate and synovitis (FIG. 10A). Such treatment also reduced destructive arthritis as evidenced by decreased pannus formation and bone erosion (FIG. 10A).

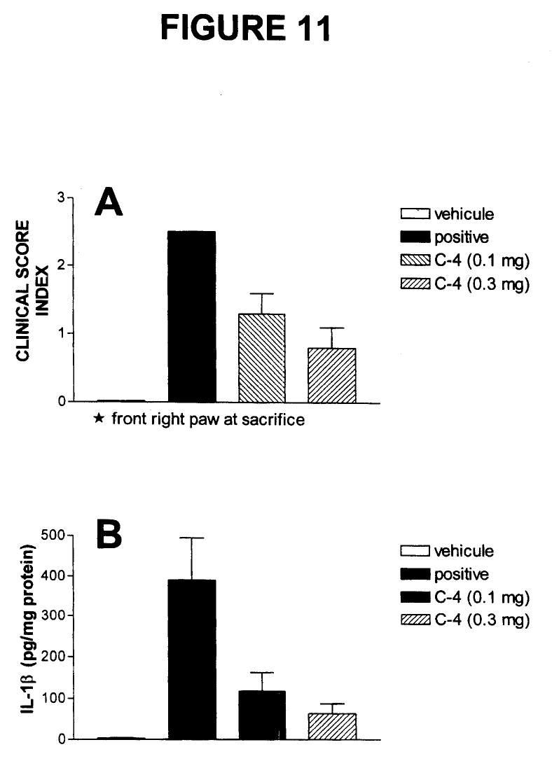

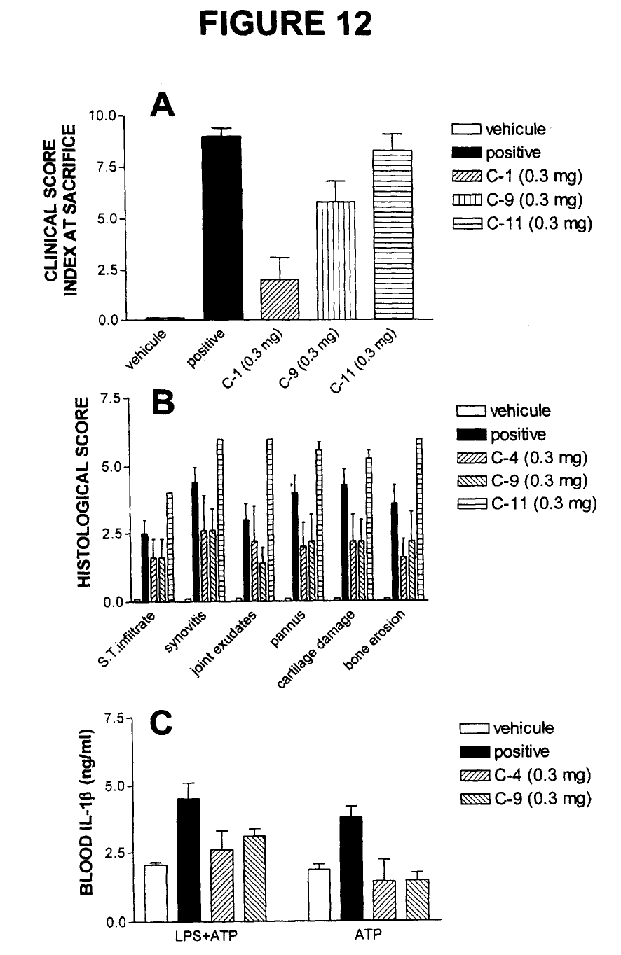

To determine whether treatment with HN compound C-4 affects the levels of IL-1.beta in joints of mice with arthritis, the right paw of mice in each group was evaluated by arthritis scoring before sacrifice and subsequently for IL-1.beta content. As illustrated in FIG. 11A and FIG. 11B treatment with HN compound C-4 decreased both the arthritis score index and the levels of IL-1.beta in the paws. This suggest that reduction of inflammatory arthritis by HN compound C-4 is related to its capacity to inhibit IL-1.beta in joints of treated mice. Reduction of the clinical and histological scores of arthritis in this mouse model was not restricted to HN compound C-4. As shown in FIG. 12A and FIG. 12B, parallel groups of mice treated with either the linear peptide C-1 (0.3 mg) or the cyclic peptide C-9 (0.3 mg) displayed similar reduction in arthritis and histological score indexes.

In contrast the non-peptide compound C-11, the 2S isomeric form of C-12 which did not inhibit IL-1.beta release in vivo in mice primed with LPS and challenged with ATP (FIG. 6B) did not reduce the arthritis and histological score indexes (FIG. 12A and FIG. 12B). Therefore, inhibition of experimental arthritis by C-1. C-4 and C-9 is related to their inherent chemical composition and correlates with their ability to inhibit IL-1.beta release in mice primed with LPS and challenged with ATP. On that basis, HN linear peptides, cyclic peptides and non-peptides are likely to reduce the symptoms of arthritis.

To determine whether C-4 and C-9 treatment modulate P2X₇ receptor function in inflammatory arthritis, P2X₇ receptor-stimulated −IL release was examined ex vivo using a blood-based IL-1.beta assay. Requirement for ATP as a secretion stimulus in LPS-activated blood has been demonstrated and this has been linked to P2X₇ receptor activation (Perregaux et al. (2000) J. Immunol. 165:4615-4623). Blood obtained from mice in various groups was activated with LPS for 3 h followed by incubation with ATP for 2 h. FIG. 12C illustrates that blood from mice with positive score of arthritis released higher levels of 1-1.beta in plasma following a two-step stimulation with LPS and ATP. Similarly, blood from arthritic mice released higher levels of IL-1.beta in response to ATP stimulation indicating that peripheral cells in blood of these animals are already primed and ready to respond to the secretion stimulus (FIG. 12C). In contrast, i.p. treatment of mice with either HN linear peptide C-4 or cyclic peptide C-9 inhibit LPS+ATP- as well as ATP-induced IL-1.beta release in peripheral blood. Therefore reduction of arthritis by HN compounds C-4 and C-9 is related to their capacity to inhibit P2X₇ receptor-induced IL-1.beta release in the periphery.

All citations are hereby incorporated by reference.

The present invention has been described with regard to one or more embodiments. However it will be apparent to persons skilled in the art that a number of variations and modifications can be made without departing from the scope of the invention as defined in the claims.

What is claimed is:

1. A method for preventing or reducing the activity of P2X₇ receptor comprising: contacting a cell which expresses said P2X₇ receptor, with a P2X₇ receptor agonist in the presence and absence of Histogranin compounds, and wherein said cell exhibits reduced ATP-stimulated IL-1. beta, IL-1. alpha, IL-18 or IL-6, or reduced ATP-stimulated cell permeabilization, or reduced ATP-stimulated blebbing/apoptosis/cytotoxicity or reduced ATP-stimulated translocation of membrane phosphatidylserine (PS) (PS FLIP) and wherein the Histogranin compound is selected from the group consisting of H—R₁-Gln-Gly-Arg-R₂—CO—R₃ wherein: R₁ represents one structure selected from the group consisting of: X-Asn-Tyr-Ala-Leu-Lys-Gly, X being an hydroxyl-containing amino acid; Y-Asn-Tyr-Ala-Leu-Lys-Gly, Y being a hydrocarbon side chain-containing amino acid; Z-Asn-Tyr-Ala-Leu-Lys-Gly, Z being an aromatic amino acid; W-Asn-Tyr-Ala-Leu-Lys-Gly, W being a sulfur-containing amino acid; U-Asn-Tyr-Ala-Leu-Lys-Gly; Ser-U-Tyr-Ala-Leu-Lys-Gly; Ser-Asn-Tyr-Ala-Leu-Lys-U; U-Tyr-Ala-Leu-Lys-Gly; Asn-Tyr-Ala-Leu-Lys-U; Tyr-Ala-Leu-U-Gly; Ala-Leu-U-Gly; Leu-U-Gly; U-Gly; and Val-Val-Tyr-Ala-Leu-Lys-U-, U being a basic amino acid; R₂ represents one structure selected from the group consisting of: a single covalent bond (no intervening amino acids); Thr-Leu; Thr-Leu-Tyr-Gly-Phe; Thr-Leu-Tyr-Gly-Phe-Cys and Thr-Leu-Tyr-Gly-Phe-Gly-Gly; and R₃ represents a radical selected from the group consisting of —OH and —NH₂;
the compound of Formula I 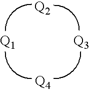
wherein:
Q₁ represents glycine alanine, valine, leucine, isoleucine, lysine, histidine, or arginine;
Q₂ represents asparagine or glutamine;
Q₃ represents glycine, alanine, valine, leucine, isoleucine, phenylalamine, tryptophan, or tyrosine; and
Q₄ represents lysine, arginine, or histidine;
pseudopeptide analogues thereof wherein one or more of the carbonyl groups of the peptide linkage is replaced by —C(═S)— or by —CH₂—, and/or wherein one or more of the amide bonds, —C(O)—NH—, is replaced by the retro-verso form, —NH—C(O)—, thereof;
the compound of Formula II, Formula III or Formula IV 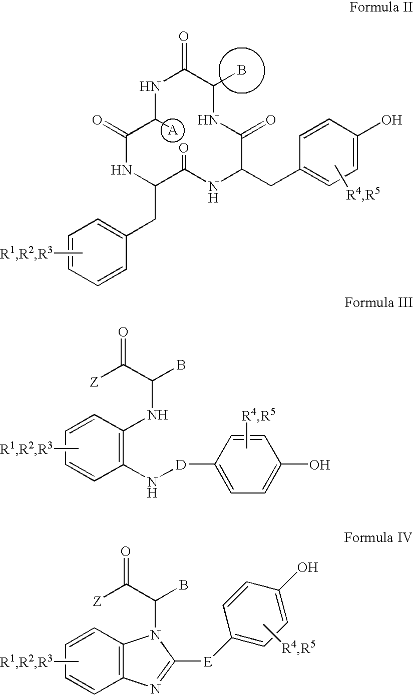
wherein:
A is -hydrogen, —(C₁-C₈) alkyl or —(C₁-C₈) alkyl substituted by hydroxy;
B is —(C₁-C₆) alkylguanidino, —(C₁-C₆) alkyl (4-imidazolyl), —(C₁-C₆) alkylamino,
p-aminophenylalkyl (C₁-C₆)—, p-guanidinophenylalkyl (C₁-C₆)— or 4-pyridinylalkyl (C₁-C₆)—;
D is —(CO)—, —(CO)—(C₁-C₆) alkylene or —(C₁-C₆) alkylene;
E is a single bond or —(C₁-C₆) alkylene;
Z is —NH₂, —NH—(C₁-C₆) alkylcarboxamide, —NH—(C₁-C₆) alkyl, —NH-benzyl, —NH-cyclic (C₅-C₇) alkyl, —NH-2-(1-piperidyl)ethyl, —NH-2-(1-pyrrolidyl)ethyl, —NH-2-(1-pyridyl)ethyl, —NH-2-(morpholino) ethyl, -morpholino, -piperidyl, —OH, —(C₁-C₆) alkoxy, —O-benzyl or —O-halobenzyl;
R¹, R² and R³ are, independent of one another, -hydrogen, -arylcarbonylamino, —(C₁-C₆) alkoylamino, —(C₁-C₆) alkylamino, —(C₁-C₆) alkyloxy, —(C₁-C₆) alkylaminocarbonyl, -carboxy, —OH, -benzoyl, -p-halogenobenzoyl, -methyl. —S-(2,4-dinitrophenyl), —S-(3-nitro-2-pyridinesulfenyl), -sulfonyl, -trifluoromethyl, —(C₁-C₆) alkylaminocarbonylamino, -halo or -amino;
R⁴ and R⁵ are, independent of one another, -hydrogen, —(C₁-C₆)alkyl, -methyloxy,
-nitro, -amino, -arylcarbonylamino, —(C₁-C₆) alkoylamino, —(C₁-C₆) alkylamino, -halo or —OH;
the compound of Formula V 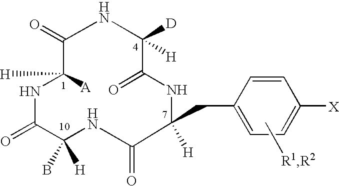
wherein:
Carbon atoms in positions 1, 4, 7 and 10 can be under the configurations S or R, but preferably S, S, S and R, respectively.
“A” is hydrogen, —(C₁-C₈)alkyl, —(C₁-C₈)alkyl substituted by hydroxyl or —(C₁-C₈)alkyl substituted by sulfur;
“B” is —(C₁-C₆)alkylguanidino, —(C₁-C₆)alkyl(4-imidazolyl) or (C₁-C₆)alkylamino;
“D” is H, methyl, —(C₁-C₈)alkyl, —(C₁-C₈)alkyl substituted by hydroxyl or —(C₁-C₈)alkyl substituted by sulphur;
R¹ and R² are, independent of one another, hydrogen, —(C₁-C₆)alkyl, methyloxy, nitro, amino, arylcarbonylamino. (C₁-C₆)alkoylamino, (C₁-C₆)alkylamino, halo or hydroxy.
“X” is hydrogen, hydroxyl or halogen;
and pharmaceutical acceptable salts and esters thereof.

2. A method of screening a test compound for its ability to modulate the inhibition of Histogranin compounds on the activity of P2X₇ receptor comprising: contacting a cell which expresses said P2X₇ receptor, with a P2X₇ receptor agonist, and a Histogranin compound, and assaying for an alteration in the activity of said P2X₇ receptor in the presence of said test compound and wherein a reduction or increase in the activity of said P2X₇ receptor being indicative of a test compound that modulates Histogranin inhibition of P2X₇ receptor activity and wherein the Histogranin compounds are as defined in claim 1.

3. The method of claim 2, wherein said P2X₇ receptor activity is selected from the group consisting of: ATP-stimulated IL-1 beta. IL-1. alpha, IL-18 or IL-6; reduced ATP-stimulated cell permeabilization; reduced ATP-stimulated blebbing; reduced ATP-stimulated apoptosis; reduced ATP-stimulated cytotoxicity; and reduced ATP-stimulated translocation of membrane phosphatidylserine (PS FLIP).

4. A method for reducing the activity of P2X₇ receptor in an animal comprising: administering a therapeutically effective amount of a Histogranin compound or a pharmaceutically acceptable salt or solvate thereof, wherein said Histogranin compound is as defined in claim 1.

5. The method of claim 4, wherein said P2X₇ receptor activity is selected from the group consisting of: ATP-stimulated IL-1. beta. IL-1. alpha IL-18 or IL-6; reduced ATP-stimulated cell permeabilization; reduced ATP-stimulated blebbing; reduced ATP-stimulated apoptosis; reduced ATP-stimulated cytotoxicity; and reduced ATP-stimulated translocation of membrane phosphatidylserine (PS FLIP).

6. The method of claim 4, wherein the compound is administered centrally or peripherally.

7. The method of claim 4, wherein the compound is administered in admixture with a pharmaceutically acceptable adjuvant, carrier, diluent or excipient.

8. The method according to claim 4, wherein the compound is administered topically in the form of solutions, suspensions, aerosols and dry powder formulations; systemically in the form of tablets, capsules, syrup, powders or granules; by parenteral administration in the form of solutions or suspensions; by subcutaneous administration; by rectal administration in the form of suppositories; or transdermally.

9. The method according to claim 4, wherein said animal is a patient with a disease associated with excessive production of IL-1 selected from the group consisting of: rheumatoid arthritis, osteoarthritis, chronic obstructive pulmonary disease, asthma, inflammatory bowel disease including both Crohn's disease and ulcerative colitis, atherosclerosis, multiple sclerosis. Alzheimer's and stroke.

10. The method according to claim 4, wherein said animal is a patient with a disease associated with excessive production of IL-18 selected from the group consisting of: rheumatoid arthritis, mechanical hypernociception, osteoarthritis, Crohn's disease, sepsis, hepatitis C virus infection and type 1 diabetes.

11. The method of claim 4, wherein said animal is a patient with a disease characterized by tissue injury and cell death, selected from the group consisting of: stroke, brain trauma, epilepsy, Alzheimer's, Parkinson's, motor neurone disease and transmissible spongiform encephalopathies.

12. A method for the treatment of rheumatoid arthritis in a patient in need thereof comprising: administering a therapeutically effective amount of a Histogranin compound or a pharmaceutically acceptable salt or solvate thereof to said patient, wherein the severity of said arthritis is reduced and wherein the Histogranin compound is as defined in claim 1.

13. The method according to claim 12, wherein the compound is administered in admixture with a pharmaceutically acceptable adjuvant, carrier, diluent or excipient .

14. The method according to claim 12, wherein the compound is administered topically in the form of solutions, suspensions, and dry powder formulations; systemically in the form of tablets, capsules, syrup, powders or granules; by parenteral administration in the form of solutions or suspensions; by subcutaneous administration; or transdermally.

15. The method according to claim 12, wherein inflammation and synovitis related to said arthritis are reduced.

16. The method according to claim 12, wherein pannus and bone erosion related to said arthritis are reduced.

17. A compound selected from the group consisting of:
Val-Val-Tyr-Ala-Leu-Lys-Arg-Gln-Gly-(3nitro-
2-sulfenyl pyridine)Cys-Thr-Leu-Tyr-Gly-Phe.
and
Val-Val-Tyr-Thr-Leu-Lys-Arg-Gln-Gly-Arg-Thr-
Leu-Tyr-Gly-Phe.
